(19) United States
(12) Patent Application Publication
(10) Pub. No.: US 2023/0260627 A1
(43) Pub. Date: Aug. 17, 2023

(54) A REMOTE MEDICAL EXAMINATION SYSTEM AND METHOD

(71) Applicant: Tyto Care LTD., Netanya (IL)

(72) Inventors: Eyal BYCHKOV, Hod Hasharon (IL); David GILAD, Even Yehuda (IL); Zvi KLEINER, Raanana (IL); Dan RIMON, (IL); Yuval GLICK, Bitzaron (IL)

(21) Appl. No.: 17/997,935

(22) PCT Filed: Apr. 27, 2021
(86) PCT No.: PCT/IL2021/050484

§ 371 Date: Nov. 4, 2022

Related U.S. Application Data
(60) Provisional application No. 63/069,057, filed on Aug. 23, 2020.
(60) Provisional application No. 63/020,792, filed on May 6, 2020.
(60) Provisional application No. 63/145,018, filed on Feb. 3, 2021.

(51) Int. Cl. G16H 30/20; G06T 7/70; A61F 13/38; A61F 13/84

(52) U.S. Cl. CPC G16H 30/20; G06T 7/70; A61F 13/38; A61F 13/84; A61F 2013/8497

(57) ABSTRACT

A medical data acquisition device comprising a processing circuitry, the processing circuitry configured to: operate a camera to acquire at least one image during a process for collecting a sample by a tip of a swab making contact with a required contact area on, or within, a body of a patient; analyze the image to determine one or more parameters, the parameters including at least one of (a) a position of the swab with respect to the required contact area, or (b) an orientation of the swab with respect to the required contact area; and perform at least one action based on comparison of the one or more parameters with a positioning criterion.

### TECHNICAL FIELD

The invention relates to a remote medical examination system and method.

### BACKGROUND

Wrong specimen collection from a patient and preanalytical errors occurring after collecting the specimen are a substantial source of medical errors that can jeopardize patient safety. Specimen collection and labeling errors have significant consequences for patient care, for healthcare management and for increasing costs that are often unaccounted for. Proper specimen collection and labeling practices are critical components of effective and accurate patient identification and patient care. During initial training, in-services and routine patient encounters, healthcare professionals can be expected to perform work properly. But when limited resources become an issue, even the best-trained, experienced staff member may deviate from proper and generally accepted practices. Moreover, in the field of telemedicine wherein clinical health care is provided from a distance, the medical practitioners rely on medical data provided by the patient himself (by voice and/or text) and on laboratory tests of specimen(s) acquired by medical data acquisition device(s) that are designed to collect specimen(s) from the patient's body. In such cases, the person collecting the specimen from the patient (e.g. a user of the medical data acquisition device(s), sometimes the patient himself) is not medically trained and can face a great challenge in collecting an accurate specimen from a correct location of the patient's body.

Recently home testing kits have greatly expanded as some of them do not require a prescription and can be purchased and used by anyone at home. Despite their benefits, such as convenience and privacy for example, they do not replace professional medical diagnosis, as they are generally only initial indicators of disease and may need a confirmatory test or another control mechanism. Moreover, home testing kits usually require the user to follow some procedural steps necessary to perform the test. If the user deviates from these procedural steps the testing kit can give misleading results.

In order to provide the patient with an accurate medical diagnosis it is necessary to ensure that the appropriate specimen type is collected and placed in the appropriate specimen container at the right time from the correct patient.

Therefore, there is a growing need to provide a new remote medical examination system and method.

### GENERAL DESCRIPTION

In accordance with a first aspect of the presently disclosed subject matter, there is provided a medical data acquisition device comprising a processing circuitry, the processing circuitry configured to: operate a camera to acquire at least one image during a process for collecting a sample by a tip of a swab making contact with a required contact area on, or within, a body of a patient; analyze the image to determine one or more parameters, the parameters including at least one of (a) a position of the swab with respect to the required contact area, or (b) an orientation of the swab with respect to the required contact area; and perform at least one action based on comparison of the one or more parameters with a positioning criterion.

In some cases, the camera is comprised within the medical data acquisition device and wherein the medical data acquisition device further comprises a mechanical connection for connecting the swab so that images captured by the camera include at least a part of the swab upon the swab being connected to the medical data acquisition device via the mechanical connection.

In some cases, the medical data acquisition device is a smartphone and wherein the mechanical connection is an add-on connectable to the smartphone for enabling positioning of the swab so that images captured by the camera include at least the part of the swab upon the swab being connected to the medical data acquisition device via the mechanical connection.

In some cases, the swab includes one or more scale mark and/or graduation marks enabling determination of a penetration depth of the swab into a body cavity of the patient.

In some cases, the swab includes one or more rotation determination markings enabling determination of rotation of the swab around an axis of rotation.

In some cases, the rotation determination markings include two non-parallel lines.

In some cases, the medical data acquisition device further comprises a mechanical connection for connecting the swab and wherein the camera is external to the medical data acquisition device and movable irrespective of the swab upon the swab being connected to the medical data acquisition device via the mechanical connection.

In some cases, upon the comparison of the one or more parameters with the positioning criterion indicating that the swab is not at a desired spatial position with respect to the required contact area, the action is one or more of: alerting a user of the medical data acquisition device, or marking the sample as invalid.

In some cases, upon the comparison of the one or more parameters with the positioning criterion indicating that the swab is at a desired spatial position with respect to the required contact area, the action is one or more of: providing a user of the medical data acquisition device with a successful positioning indication, or marking the sample as valid.

In some cases, the required contact area is within a cavity of the body and wherein the positioning criterion depends on a length of an inserted part of the swab inserted into the cavity.

In some cases, the length of the inserted part of the swab inserted into the cavity depends on parameters of the patient.

In some cases, the parameters include one or more of: age, height, gender, a structure of the cavity.

In some cases, the cavity is a nostril.

In some cases, the processing circuitry is further configured to provide a user of the medical data acquisition device with a successful sampling indication upon meeting a sampling criterion.

In some cases, the processing circuitry is further configured to: operate the camera to acquire a sequence of images during the process for collecting the sample; analyze the sequence of images to determine one or more navigation parameters, the navigation parameters including at least one of: (a) a navigation position of the swab with respect to the required contact area, or (b) a navigation orientation of the swab with respect to the required contact area; and based on the navigation parameters, provide a user of the medical data acquisition device with navigation instructions for navigating the tip of the swab to contact the required contact area.

In some cases, the processing circuitry is further configured to identify, within the at least one image, the required contact area, or a cavity leading to the required contact area.

In some cases, the required contact area is determined by an operator of the medical data acquisition device.

In some cases, the operator is located at a remote location, remote from the patient.

In some cases, the processing circuitry is further configured to: operate the camera to acquire at least one sample placement image after collecting the sample, the sample placement image including one or more receptacles, each including a respective reagent, and each having an identifier enabling determination of a type of the respective reagent; analyze the sample placement image to identify a given receptacle of the receptacles, being the receptacle associated with a reagent for testing a given medical condition of the patient; and perform one or more second actions based on results of the analysis.

In some cases, the second actions include one or more of: (a) display the sample placement image on a display with a marking of the given receptacle on the sample placement image, (b) upon the results indicating that the swab is within the given receptacle provide a user with a successful tube placement indication, (c) upon the results indicating that the swab is not within the given receptacle provide the user with a failure tube placement indication or with placement instructions for placing the swab within the given receptacle, (d) upon the results indicating that the swab is within the given receptacle provide the user with a medical diagnosis, (e) upon the results indicating that the swab is within the given receptacle provide the user with treatment instructions.

In some cases, the processing circuitry is further configured to: operate the camera to acquire at least one expiration date validation image, the expiration date validation image including one or more receptacles, each having an identifier enabling determination of an expiration date of the respective receptacle; analyze the expiration date validation image to validate an expiration date of the receptacles; and perform an expiration action upon the expiration date of one or more of the receptacles indicating expiry.

In some cases, the expiration action includes providing an expiration notification to a user.

In accordance with a second aspect of the presently disclosed subject matter, there is provided a medical data acquisition device comprising a processing circuitry, the processing circuitry configured to: operate a camera to acquire at least one image during a process for collecting a sample by a tip of the swab making contact with a required contact area on, or within, a body of a patient; analyze the at least one image to determine if the tip is in contact with a required contact area; and perform at least one action based on results of the analysis.

In some cases, the camera is comprised within the medical data acquisition device and wherein the medical data acquisition device further comprises a mechanical connection for connecting the swab so that images captured by the camera include at least the tip of the swab upon the swab being connected to the medical data acquisition device via the mechanical connection.

In some cases, the medical data acquisition device is a smartphone and wherein the mechanical connection is an add-on connectable to the smartphone for enabling positioning of the swab so that images captured by the camera include at least the tip of the swab upon the swab being connected to the medical data acquisition device via the mechanical connection.

In some cases, the swab includes one or more scale mark and/or graduation marks enabling determination of a penetration depth of the swab into a body cavity of the patient.

In some cases, the swab includes one or more rotation determination markings enabling determination of rotation of the swab around an axis of rotation.

In some cases, the rotation determination markings include two non-parallel lines.

In some cases, the medical data acquisition device further comprises a mechanical connection for connecting the swab and wherein the camera is external to the medical data acquisition device and movable irrespective of the swab upon the swab being connected to the medical data acquisition device via the mechanical connection.

In some cases, upon the determination indicating that the tip of the swab is not in contact with the required contact area, the action is one or more of: alerting a user of the medical data acquisition device, or marking the sample as invalid.

In some cases, upon the determination indicating that tip of the swab is in contact with the required contact area, the action is one or more of: providing a user of the medical data acquisition device with a successful positioning indication, or marking the sample as valid.

In some cases, the processing circuitry is further configured to analyze the image to determine the required contact area.

In some cases, the required contact area is determined by an operator of the medical data acquisition device.

In some cases, the operator is located at a remote location, remote from the patient.

In some cases, the processing circuitry is further configured to: operate the camera to acquire a sequence of images during the process for collecting the sample; analyze the images to determine one or more navigation parameters, the navigation parameters including at least one of: (a) a navigation position of the swab with respect to the required contact area, or (b) a navigation orientation of the swab with respect to the required contact area; and based on the navigation parameters, provide a user of the medical data acquisition device with navigation instructions for navigating the tip of the swab to contact the required contact area.

In some cases, the processing circuitry is further configured to provide a user of the medical data acquisition device with a successful positioning indication upon the tip being in contact with the required contact area.

In some cases, the processing circuitry is further configured to provide a user of the medical data acquisition device with a successful sampling indication upon meeting a sampling criterion.

In some cases, the processing circuitry is further configured to: operate the camera to acquire at least one sample placement image after collecting the sample, the sample placement image including one or more receptacles, each including a respective reagent, and each having an identifier enabling determination of a type of the respective reagent; analyze the sample placement image to identify a given receptacle of the receptacles, being the receptacle associated with a reagent for testing a given medical condition of the patient; and perform one or more second actions based on results of the analysis.

In some cases, the second actions include one or more of: (a) display the sample placement image on a display with a marking of the given receptacle on the sample placement image, (b) upon the results indicating that the swab is within the given receptacle provide the user with a successful tube placement indication, (c) upon the results indicating that the swab is not within the given receptacle provide the user with a failure tube placement indication or with placement instructions for placing the swab within the given receptacle, (d) upon the results indicating that the swab is within the given receptacle provide the user with a medical diagnosis, (e) upon the results indicating that the swab is within the given receptacle provide the user with treatment instructions.

In some cases, the processing circuitry is further configured to: operate the camera to acquire at least one expiration date validation image, the expiration date validation image including one or more receptacles, each having an identifier enabling determination of an expiration date of the respective receptacle; analyze the expiration date validation image to validate an expiration date of the receptacles; and perform an expiration action upon the expiration date of one or more of the receptacles indicating expiry.

In some cases, the expiration action includes providing an expiration notification to a user.

In accordance with a third aspect of the presently disclosed subject matter, there is provided a method comprising: operating, by a processing circuitry, a camera, to acquire at least one image during a process for collecting a sample by a tip of a swab making contact with a required contact area on, or within, a body of a patient; analyzing, by the processing circuitry, the image to determine one or more parameters, the parameters including at least one of (a) a position of the swab with respect to the required contact area, or (b) an orientation of the swab with respect to the required contact area; and performing, by the processing circuitry, at least one action based on comparison of the one or more parameters with a positioning criterion.

In some cases, the camera is comprised within a medical data acquisition device and wherein the medical data acquisition device comprises a mechanical connection for connecting the swab so that images captured by the camera include at least a part of the swab upon the swab being connected to the medical data acquisition device via the mechanical connection,

In some cases, the medical data acquisition device is a smartphone and wherein the mechanical connection is an add-on connectable to the smartphone for enabling positioning of the swab so that images captured by the camera include at least the part of the swab upon the swab being connected to the medical data acquisition device via the mechanical connection.

In some cases, the swab includes one or more scale mark and/or graduation marks enabling determination of a penetration depth of the swab into a body cavity of the patient.

In some cases, the swab includes one or more rotation determination markings enabling determination of rotation of the swab around an axis of rotation.

In some cases, the rotation determination markings include two non-parallel lines.

In some cases, the swab is connected to a medical data acquisition device via a mechanical connection and wherein the camera is external to the medical data acquisition device and movable irrespective of the swab upon the swab being connected to the medical data acquisition device via the mechanical connection.

In some cases, upon the comparison of the one or more parameters with the positioning criterion indicating that the swab is not at a desired spatial position with respect to the required contact area, the action is one or more of: alerting a user of the medical data acquisition device, or marking the sample as invalid.

In some cases, upon the comparison of the one or more parameters with the positioning criterion indicating that the swab is at a desired spatial position with respect to the required contact area, the action is one or more of: providing a user of the medical data acquisition device with a successful positioning indication, or marking the sample as valid.

In some cases, the required contact area is within a cavity of the body and wherein the positioning criterion depends on a length of an inserted part of the swab inserted into the cavity.

In some cases, the length of the inserted part of the swab inserted into the cavity depends on parameters of the patient.

In some cases, the parameters include one or more of: age, height, gender, or a structure of the cavity.

In some cases, the cavity is a nostril.

In some cases, the method further comprises providing, by the processing circuitry, a user of the medical data acquisition device with a successful sampling indication upon meeting a sampling criterion.

In some cases, the method further comprises: operating, by the processing circuitry, the camera to acquire a sequence of images during the process for collecting the sample; analyzing, by the processing circuitry, the sequence of images to determine one or more navigation parameters, the navigation parameters including at least one of: (a) a navigation position of the swab with respect to the required contact area, or (b) a navigation orientation of the swab with respect to the required contact area; and based on the navigation parameters, providing, by the processing circuitry, a user of the medical data acquisition device with navigation instructions for navigating the tip of the swab to contact the required contact area.

In some cases, the method further comprises identifying, by the processing circuitry, within the at least one image, the required contact area, or a cavity leading to the required contact area.

In some cases, the required contact area is determined by an operator of the medical data acquisition device.

In some cases, the operator is located at a remote location, remote from the patient.

In some cases, the method further comprises: operating, by the processing circuitry, the camera to acquire at least one sample placement image after collecting the sample, the sample placement image including one or more receptacles, each including a respective reagent, and each having an identifier enabling determination of a type of the respective reagent; analyzing, by the processing circuitry, the sample placement image to identify a given receptacle of the receptacles, being the receptacle associated with a reagent for testing a given medical condition of the patient; and performing, by the processing circuitry, one or more second actions based on results of the analysis.

In some cases, the second actions include one or more of: (a) display the sample placement image on a display with a marking of the given receptacle on the sample placement image, (b) upon the results indicating that the swab is within the given receptacle provide the user with a successful tube placement indication, (c) upon the results indicating that the swab is not within the given receptacle provide the user with a failure tube placement indication or with placement instructions for placing the swab within the given receptacle, (d) upon the results indicating that the swab is within the given receptacle provide the user with a medical diagnosis, (e) upon the results indicating that the swab is within the given receptacle provide the user with treatment instructions.

In some cases, the method further comprises: operating, by the processing circuitry, the camera to acquire at least one expiration date validation image, the expiration date validation image including one or more receptacles, each having an identifier enabling determination of an expiration date of the respective receptacle; analyzing, by the processing circuitry, the expiration date validation image to validate an expiration date of the receptacles; and performing, by the processing circuitry, an expiration action upon the expiration date of one or more of the receptacles indicating expiry.

In some cases, the expiration action includes providing an expiration notification to a user.

In accordance with a fourth aspect of the presently disclosed subject matter, there is provided a method comprising: operating, by a processing circuitry, a camera, to acquire at least one image during a process for collecting a sample by a tip of a swab making contact with a required contact area on, or within, a body of a patient; analyzing, by the processing circuitry, the at least one image to determine if the tip is in contact with a required contact area; and performing, by the processing circuitry, at least one action based on results of the analysis.

In some cases, the camera is comprised within a medical data acquisition device and wherein the medical data acquisition device comprises a mechanical connection for connecting the swab so that images captured by the camera include at least the tip of the swab upon the swab being connected to the medical data acquisition device via the mechanical connection.

In some cases, the medical data acquisition device is a smartphone and wherein the mechanical connection is an add-on connectable to the smartphone for enabling positioning of the swab so that images captured by the camera include at least the part of the swab upon the swab being connected to the medical data acquisition device via the mechanical connection.

In some cases, the swab includes one or more scale mark and/or graduation marks enabling determination of a penetration depth of the swab into a body cavity of the patient.

In some cases, the swab includes one or more rotation determination markings enabling determination of rotation of the swab around an axis of rotation.

In some cases, the rotation determination markings include two non-parallel lines.

In some cases, the swab is connected to a medical data acquisition device via a mechanical connection and wherein the camera is external to the medical data acquisition device and movable irrespective of the swab upon the swab being connected to the medical data acquisition device via the mechanical connection.

In some cases, upon the determination indicating that the tip of the swab is not in contact with the required contact area, the action is one or more of: alerting a user of the medical data acquisition device, or marking the sample as invalid.

In some cases, upon the determination indicating that tip of the swab is in contact with the required contact area, the action is one or more of: providing a user of the medical data acquisition device with a successful positioning indication, or marking the sample as valid.

In some cases, the method further comprises analyzing, by the processing circuitry, the image to determine the required contact area.

In some cases, the required contact area is determined by an operator of the medical data acquisition device.

In some cases, the operator is located at a remote location, remote from the patient.

In some cases, the method further comprises: operating, by the processing circuitry, the camera to acquire a sequence of images during the process for collecting the sample; analyzing, by the processing circuitry, the images to determine one or more navigation parameters, the navigation parameters including at least one of: (a) a navigation position of the swab with respect to the required contact area, or (b) a navigation orientation of the swab with respect to the required contact area; and based on the navigation parameters, providing, by the processing circuitry, a user of the medical data acquisition device with navigation instructions for navigating the tip of the swab to contact the required contact area.

In some cases, the method further comprises providing, by the processing circuitry, a user of the medical data acquisition device with a successful positioning indication upon the tip being in contact with the required contact area.

In some cases, the method further comprising providing, by the processing circuitry, a user of the medical data acquisition device with a successful sampling indication upon meeting a sampling criterion.

In some cases, the method further comprises: operating, by the processing circuitry, the camera to acquire at least one sample placement image after collecting the sample, the sample placement image including one or more receptacles, each including a respective reagent, and each having an identifier enabling determination of a type of the respective reagent; analyzing, by the processing circuitry, the sample placement image to identify a given receptacle of the receptacles, being the receptacle associated with a reagent for testing a given medical condition of the patient; and performing, by the processing circuitry, one or more second actions based on results of the analysis.

In some cases, the second actions include one or more of: (a) display the sample placement image on a display with a marking of the given receptacle on the sample placement image, (b) upon the results indicating that the swab is within the given receptacle provide the user with a successful tube placement indication, (c) upon the results indicating that the swab is not within the given receptacle provide the user with a failure tube placement indication or with placement instructions for placing the swab within the given receptacle, (d) upon the results indicating that the swab is within the given test tube provide the user with a medical diagnosis, (e) upon the results indicating that the swab is within the given receptacle provide the user with treatment instructions.

In some cases, the method further comprises: operating, by the processing circuitry, the camera to acquire at least one expiration date validation image, the expiration date validation image including one or more receptacles, each having an identifier enabling determination of an expiration date of the respective receptacle; analyzing, by the processing circuitry, the expiration date validation image to validate an expiration date of the receptacles; and performing, by the processing circuitry, an expiration action upon the expiration date of one or more of the receptacles indicating expiry.

In some cases, the expiration action includes providing an expiration notification to a user.

In accordance with a fifth aspect of the presently disclosed subject matter, there is provided a non-transitory computer readable storage medium having computer readable program code embodied therewith, the computer readable program code, executable by at least one processing circuitry to perform a method comprising: operating, by the processing circuitry, a camera, to acquire at least one image during a process for collecting a sample by a tip of the swab making contact with a required contact area on, or within, a body of a patient; analyzing, by the processing circuitry, the image to determine one or more parameters, the parameters including at least one of (a) a position of the swab with respect to the required contact area, or (b) an orientation of the swab with respect to the required contact area; and performing, by the processing circuitry, at least one action based on comparison of the one or more parameters with a positioning criterion.

In accordance with a sixth aspect of the presently disclosed subject matter, there is provided a non-transitory computer readable storage medium having computer readable program code embodied therewith, the computer readable program code, executable by at least one processing circuitry to perform a method comprising: operating, by a processing circuitry, a camera, to acquire at least one image during a process for collecting a sample by the tip of the swab making contact with a required contact area on, or within, a body of a patient; analyzing, by the processing circuitry, the at least one image to determine if the tip is in contact with a required contact area; and performing, by the processing circuitry, at least one action based on results of the analysis.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is a block diagram schematically illustrating one example of a system for performing a medical examination of a patient by a remote medical practitioner, in accordance with the presently disclosed subject matter;

FIG. 2 is a flowchart illustrating one example of a sequence of operations carried out for performing a remote examination, in accordance with the presently disclosed subject matter;

FIG. 3 is a flowchart illustrating another example of a sequence of operations carried out for performing a remote examination, in accordance with the presently disclosed subject matter;

FIG. 4 is a flowchart illustrating an example of a sequence of operations carried out for providing navigation guidance during performance of a remote examination, in accordance with the presently disclosed subject matter;

FIG. 5 is a flowchart illustrating an example of a sequence of operations carried out for sample placement verification, in accordance with the presently disclosed subject matter;

FIG. 6 is a flowchart illustrating an example of a sequence of operations carried out for expiration date validation, in accordance with the presently disclosed subject matter;

FIG. 7a is an illustration of acquiring a sample from a patient's nostril using a swab not connected to the medical data acquisition device, in accordance with the presently disclosed subject matter;

FIG. 7b is an illustration of acquiring a sample from a patient's nostril using a swab connected to the medical data acquisition device, in accordance with the presently disclosed subject matter;

FIG. 8a is an illustration of acquiring a sample from a patient's throat using a swab not connected to the medical data acquisition device, in accordance with the presently disclosed subject matter;

FIG. 8b is an illustration of acquiring a sample from a patient's throat using a swab connected to the medical data acquisition device, in accordance with the presently disclosed subject matter;

FIG. 9 is an illustration of verification of correct chemical compounds addition into a receptacle, in accordance with the presently disclosed subject matter; and

FIG. 10 is an illustration of a swab with markings enabling determination of penetration depth and rotation, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “receiving”, “analyzing”, “operating”, “performing”, “identifying”, “providing”, “sending” or the like, include action and/or processes of a computer that manipulate and/or transform data into other data, said data represented as physical quantities, e.g. such as electronic quantities, and/or said data representing the physical objects. The terms “computer”, “processor”, “processing resource” and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

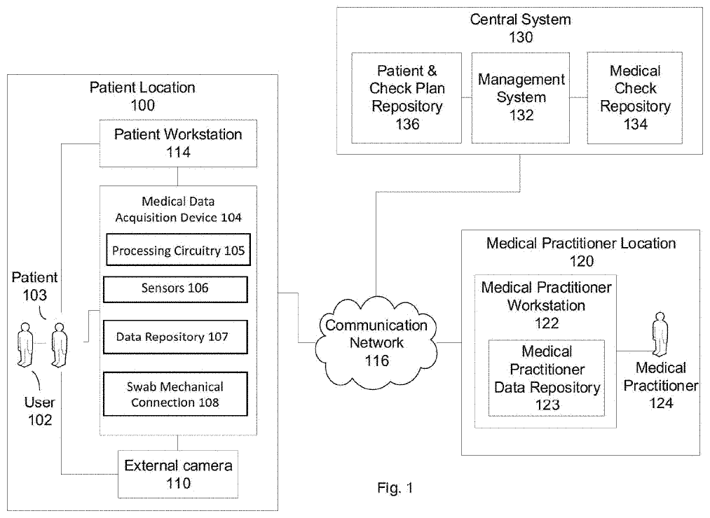

In embodiments of the presently disclosed subject matter, fewer, more and/or different stages than those shown in FIGS. 2-6 may be executed. In embodiments of the presently disclosed subject matter one or more stages illustrated in FIGS. 2-6 may be executed in a different order and/or one or more groups of stages may be executed simultaneously. FIG. 1 illustrates a general schematic of the system architecture in accordance with an embodiment of the presently disclosed subject matter. Each module in FIG. 1 can be made up of any combination of software, hardware and/or firmware that performs the functions as defined and explained herein. The modules in FIG. 1 may be centralized in one location or dispersed over more than one location. In other embodiments of the presently disclosed subject matter, the system may comprise fewer, more, and/or different modules than those shown in FIG. 1.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

Bearing this in mind, attention is drawn to FIG. 1, a block diagram schematically illustrating one example of a system for performing a medical examination of a patient by a remote medical practitioner, in accordance with the presently disclosed subject matter. A user 102 and a patient 103 (a person or an animal whose medical examination is required) are located at patient location 100, and a medical practitioner 124 is located at a medical practitioner location 120, remote from the patient location 100. In fact, according to the presently disclosed subject matter, the medical practitioner 124 is located at the medical practitioner location 120 that is remote from the patient location 100 so that the medical practitioner 124 does not have direct access to the patient 103 (e.g. it is not located in the same room with the patient 103, nor in any other form of vicinity thereto, so that the medical practitioner 124 cannot himself hold the medical data acquisition device 104 and place it on the body of the patient 103 for acquiring medical data therefrom). In some cases, the medical practitioner 124 can be located in a different room/floor/building/street/city/state/country/continent than the patient 103.

In light of the fact that the medical practitioner 124 is located at a different location than the patient 103, the user 102 is required to operate the medical data acquisition device 104 for acquiring medical data from the patient's 103 body. In this respect, it is to be noted that the user 102 can be the patient 103 whose medical examination is required (in such cases, even though user 102 and patient 103 are shown as separate entities in the drawings, they are in fact the same entity). In other cases, the user 102 can be another person (other than patient 103) that will operate the medical data acquisition device 104 for acquiring medical data from the patient's 103 body, as further detailed herein. In some cases, the user 102 is not a medical practitioner, i.e. the user 102 is not a person specifically trained to acquire medical data from the patient's 103 body, nor is he qualified to diagnose a medical condition of the patient 103 based on medical data acquired from the patient's body.

Attention is drawn to the components within the patient location 100:

The medical data acquisition device 104 comprises (or is otherwise associated with) at least one processing circuitry 105. Processing circuitry 105 can be one or more processing units (e.g. central processing units), microprocessors, microcontrollers (e.g. microcontroller units (MCUs)) or any other computing/processing device, which are adapted to independently or cooperatively process data for controlling relevant medical data acquisition device 104 resources and for enabling operations related to medical data acquisition device 104 resources.

Medical data acquisition device 104 can further comprise one or more sensors 106 (e.g. camera/s, microphone/s, a thermometer, depth camera/s, an otoscope, a blood pressure sensor, an electrocardiogram (ECG), an ultrasound sensor, an acoustic sensor, a blood saturation sensor, etc.), including at least one sensor capable of acquiring medical data from the patient's 103 body, based on which the medical practitioner 124 can diagnose a medical condition of the patient 103. The medical data can be, for example, body temperature, blood pressure, blood saturation, ECG measurements, audio signals (e.g. of the heart operations or of the lungs), ultrasound signals (e.g. of the heart, of the intestines, etc.), acoustic measurements, body tissue electrical resistance, hardness of body tissues, a heartrate, an image or a video recording of a body organ or a portion of a body organ (whether internal body organ or external body organ), a 3D representation of one or more body organs or portions thereof (whether internal body organ or external body organ), a blood sample analysis, urine samples, throat cultures, saliva samples, or any other parameter associated with one or more physiological characteristic of a patient, based on which diagnosis can be provided.

In some cases, medical data acquisition device 104 can further comprise a swab mechanical connection 108. The swab mechanical connection 108 is configured to connect a swab to the medical data acquisition device 104. The swab can be a handheld item that consists of a number of small pieces (e.g. one or two or more) of soft and/or absorbing material wrapped around one or both ends of a rod (also referred to herein as “tip”), and can be used for taking a small amount of substance from a body of a patient. The swab can be disposable or reusable and its material may vary based on the particular diagnostic test type to be taken (e.g. cotton swab with wooden rod, flocked or spun polyester swab with plastic shaft, etc.). Since, in some cases, the swabs may be used for collecting samples for diagnosing different medical conditions, different types of swabs can be used, such as but not limited to, cervical swab, eye swab, ear swab, nasal swab, gonorrhoea swab, etc., wherein each swab may have for example an appropriate shape, size (e.g., length), etc. Therefore, in some cases, each swab may be labelled differently in accordance with the body site from which the sample is to be taken and for this purpose each swab may have physical and/or visual identifier(s) thereon in order to enable an appropriate identification thereof, for example, by the user 102 and/or by the medical data acquisition device 104, where applicable, in accordance with the presently disclosed subject matter. Said identifier(s) may be for example and without limitation, rod shapes and/or color(s), line marks, color marks and/or labels, barcodes, rotation determination marks (e.g. rotation determination mark 1020 shown in FIG. 10) painted (or otherwise applied) on the swab, as further detailed herein, inter alia with respect to FIG. 10. Performing sample collection with a correct swab is important, as the swab site may determine, inter alia, the appropriate culture media required to target the most likely pathogens.

In addition, each swab may have an expiration date can indicate the date after which the swab should not be used for collecting a sample, and such expiration date may be monitored in a similar manner to the sample expiration date monitoring described hereinbelow with respect to FIG. 6, mutatis mutandis.

In some cases, the medical data acquisition device 104 can further comprise a tongue depressor (not shown) designed to prevent the patient's 103 tongue from touching a swab connected to the medical data acquisition device 104 via the mechanical connection 108, and/or to allow a clear line of sight for the medical data acquisition device's 104 camera to view the tip of the swab touching a desired point within the patient's 103 throat.

In some cases, medical data acquisition device 104 can further comprise, or be otherwise associated with, a data repository 107 (e.g. a database, a storage system, a memory including Read Only Memory—ROM, Random Access Memory—RAM, or any other type of memory, etc.) configured to store data, including inter alia patient-related data relating to one or more patients 103 and various medical data acquired from such patients 103 body (e.g. data acquired during a medical examination of the patients using the medical data acquisition device 104), various configuration parameters of the sensor(s) 106, check plans for patient 103 (e.g. defining medical examinations to be performed on patient 103), threshold parameters (e.g. defining required quality levels for various types of measurements), etc. In some cases, data repository 107 can be further configured to enable retrieval and/or update and/or deletion of the stored data. It is to be noted that in some cases, data repository 107 can be distributed across multiple locations, whether within the medical data acquisition device 104 and/or within patient location 100 and/or within central system 130 and/or within medical practitioner location 120 and/or elsewhere. It is to be noted, that in some cases, the relevant information relating to the patient 103 can be loaded into data repository 107 before performing medical examination of the 103 (e.g. upon beginning of a medical examination and/or periodically and/or upon an entity such as the medical practitioner 124 requesting the information).

It is to be noted that in some cases, the medical data acquisition device 104 can be a handheld device (whether proprietary or an existing product such as a smartphone), and at least the processing circuitry 105 and the sensors 106 can be comprised within a housing of the medical data acquisition device 104, that can optionally be a handheld device. In some cases, the sensors can be comprised within removably attachable units configured to be attached to the medical data acquisition device 104. In some cases, the sensors can be external to the medical data acquisition device 104 and in such cases, it may communicate with the medical data acquisition device 104 via a wired connection and/or via a wireless connection (e.g. a WiFi connection).

Another type of removably attachable units are configured to enable connection of various components to the medical data acquisition device 104. Such removably attachable units can comprise mechanical connections enabling connecting the components thereto, and by that, to the medical data acquisition device 104 (when such removably attachable units are attached to the medical data acquisition device 104). It is to be noted, in this respect, that in some cases, the swab mechanical connection 108 can optionally be such a removably attachable unit.

It is to be further noted that in some cases, medical data acquisition device 104 can further comprise one or more speakers for providing audio recordings to the user 102 (e.g. recordings of a medical practitioner 124 instructing the user 102 how to perform medical examinations, voice instructions generated by the medical data acquisition device 104 instructing the user 102 how to perform medical examinations, etc.). Medical data acquisition device 104 can further comprise a microphone for recording sounds, including voices (e.g. of the user 102 and/or patient 103), in the vicinity of the medical data acquisition device 104, e.g. during medical examinations conducted using the medical data acquisition device 104. Medical data acquisition device 104 can further comprise a display for providing visual output to the user 102 (e.g. a video recording of a remote medical practitioner 124, computer generated instructions instructing the user 102 how to perform medical examinations, indications of quality of an acquired measurement, etc.).

In some cases, medical data acquisition device 104 can communicate, directly, or indirectly, with patient workstation 114 and/or with medical practitioner workstation 122 and/or with central system 130, through communication network 116 (e.g. the Internet), via wired or wireless communication. It is to be noted that such communication can alternatively or additionally be performed utilizing other known communication alternatives, such as a cellular network, Virtual Private Network (VPN), Local Area Network (LAN), etc.

In some cases, a camera 110 can also be located at the patient location 100. Camera 110 (also referred to as “external camera 110”) is external to medical data acquisition device 104, in the sense that it is not comprised within the housing of the medical data acquisition device 104. Camera 110 is preferably movable irrespectively of medical data acquisition device 104. Camera 110 is operable to capture visible light, and to generate images or video based on light it captures. Camera 110 may additionally, or alternatively, be sensitive to other parts of the electromagnetic spectrum near the visible spectrum (e.g. to infrared radiation, such as near IR radiation). Camera 110 may be sensitive to the entire visible spectrum (e.g. a commercial-off-the-shelf camera, such as a DSLR camera, a smartphone camera, a webcam camera), or only to a part of it. In some cases, the camera 110 can be a depth camera, capable of generating a 3D representation of the examination process.

Camera 110 is oriented toward the examined patient's 103 body location, in at least some of the time during which medical data acquisition device 104 acquires medical data from the patient's 103 body. Especially, camera 110, when oriented toward the examined patient's 103 body location (as described), is operable to acquire one or more images (that can optionally form a video) which includes at least a part of the patient's 103 body and at least part of the medical data acquisition device 104 when medical data acquisition device 104 (or one or more of the sensors 106) is adjacent to the examined patient 103 body location. Accordingly, images capture by the camera 110 include at least part of the medical data acquisition device 104 and a location on the body of the patient 103 which is currently examined thereby.

In some cases, a patient workstation 114 can also be located at the patient location 100. Patient workstation 114 can be any computer, including a personal computer, a portable computer, a smartphone, a smart television (smart TV) or any other apparatus with appropriate processing capabilities, including an apparatus which can be, for example, specifically configured for that purpose. The patient workstation 114 can be operated by user 102, for receiving inputs therefrom (e.g. questions to answers, various identification information, etc.), and/or for providing output thereto (showing operational instructions for operating the medical data acquisition device 104, etc.). In some cases, patient workstation 114 can communicate with medical data acquisition device 104 and/or with medical practitioner workstation 122 and/or with central system 130, through communication network 116 (e.g. the Internet), via wired or wireless communication. It is to be noted that such communication can alternatively or additionally be performed utilizing other known communication alternatives, such as a cellular network, Virtual Private Network (VPN), Local Area Network (LAN), etc. It is to be noted that in some cases, patient workstation 114 can comprise the camera 110, and in a more specific example, patient workstation 114 can be a smartphone and camera 110 can be a camera of the smartphone. It is to be noted that in some cases, the processing resources of the patient workstation 114, or of any other computer (located at the patient location 100 or elsewhere), can perform some of the tasks described with reference to processing resource 105 of the medical data acquisition device 104.

Attention is drawn to the components within the medical practitioner location 120:

A medical practitioner workstation 122 is located at the medical practitioner location 120. Medical practitioner workstation 122 can be any computer, including a personal computer, a portable computer, a smartphone, a smart television (smart TV) or any other apparatus with appropriate processing capabilities, including an apparatus which can be, for example, specifically configured for that purpose. The medical practitioner workstation 122 can receive inputs from the medical practitioner 124 (e.g. instructions and/or questions to be provided to the user 102 and/or patient 103, etc.), and/or provide output to the medical practitioner 124 (showing the medical data acquired by the medical data acquisition device 104, etc.). In some cases, medical practitioner workstation 122 can communicate with medical data acquisition device 104 and/or patient workstation 114 and/or central system 130, through communication network 116 (e.g. the Internet), via wired or wireless communication. It is to be noted that such communication can alternatively or additionally be performed utilizing other known communication alternatives, such as a cellular network, VPN, LAN, etc. In some cases, medical practitioner workstation 122 can communicate with one or more other medical practitioner workstations 122, e.g. when a first medical practitioner operating the medical practitioner workstation 122 is interested in obtaining a second opinion, optionally relating to a certain diagnosis provided by the first medical practitioner, from another medical practitioner.

In some cases, medical practitioner workstation 122 can further comprise, or be otherwise associated with, a medical practitioner data repository 123 (e.g. a database, a storage system, a memory including Read Only Memory—ROM, Random Access Memory—RAM, or any other type of memory, etc.) configured to store data, including inter alia medical data acquired by the medical data acquisition device 104 (optionally including also various metadata relating to such medical data), and other patient-related data relating to one or more patients 103. In some cases, medical practitioner data repository 123 can be further configured to enable retrieval and/or update and/or deletion of the stored data. It is to be noted that in some cases, medical practitioner data repository 123 can be distributed across multiple locations, whether within the medical practitioner location 120 and/or within central system 130 and/or elsewhere. It is to be noted, that in some cases, the relevant information relating to a given examined patient 103 can be loaded into data repository 123 before performing medical examination of a patient 103 (e.g. upon beginning of a medical examination and/or periodically and/or upon an entity such as the medical practitioner 124 requesting the information). In some cases, the medical data can include Electronic Health Records (EHR) data relating to one or more patients 103. In some cases, the EHR data can be obtained through an interface (e.g. over the communication network 116) to a remote EHR system.

In some cases, medical practitioner system 122 can communicate with patient workstation 144 and/or with medical data acquisition device 104 and/or with central system 130, through communication network 116 (e.g. the Internet), via wired or wireless communication. It is to be noted that such communication can alternatively or additionally be performed utilizing other known communication alternatives, such as a cellular network, Virtual Private Network (VPN), Local Area Network (LAN), etc.

In some cases, a central system 130 can exist, for allowing a distributed approach in which medical data and/or other patient-related data can be received by the central system 130 from multiple patient locations 100 and transferred by it to multiple medical practitioner locations 120. Thus, in case the transmitted medical data and/or other patient-related data is received at central system 130, it can be stored in medical check repository 134 and management system 132 can transmit it to a specific medical practitioner location 120 (e.g. via communication network 116 such as the Internet). In some cases, management system 132 can also manage other processes such as, subscribing patients, planning scheduling of patients to available medical practitioners, etc.

It is to be noted that central system 130 is optional to the solution and that central system 130 can be part of the medical practitioner workstation 122. In addition, the communication between the patient workstation 114 and/or the medical data acquisition device 104, and the medical practitioner workstation 122 can be performed directly without the use of or need for a central system 130.

In those cases where a central system 130 exists, it can comprise patient & check plan repository 136 in which various patient-related data, relating to one or more patients 103, is maintained. Such patient-related data can include, for example, patient identification number, patient name, patient age, patient contact details, patient medical record data (such as the patients EHR, information of patient's diseases, sensitivities to medicines, etc.), check plans data (as further detailed below), etc. Central system 130 can further comprise a medical check repository 134 in which one or more of the following can be stored: (a) medical data acquired by medical data acquisition device 104 (optionally including also various metadata relating to such medical data), (b) user-provided data, provided by the user 102, e.g. using the patient workstation 114, including type-ins and/or voice recording and/or additional info provided by user 102 and relating to the patient 103, and (c) diagnosis data provided by a medical practitioner diagnosing the patient 103. The medical data and/or the user-provided data, can include, for example, voice recordings and/or video recordings and/or values of one or more of the following parameters: body temperature, blood pressure, blood saturation, electrocardiogram (ECG) measurements, audio signals (e.g. of the heart operations or of the lungs), ultrasound signals (e.g. of the heart, of the intestines, etc.), acoustic measurements, body tissue electrical resistance, hardness of body tissues, a heartrate, an image or a video recording of a body organ or a portion of a body organ (whether internal body organ or external body organ), a blood sample analysis, a 3D representation of one or more body organs or portions thereof (whether internal body organ or external body organ), urine samples, throat cultures, saliva samples, or any other parameter associated with one or more physiological characteristic of a patient, based on which diagnosis can be provided. In some cases, one or more of the parameter values can be associated with metadata, such as a timestamp indicative of the time in which the parameter value was acquired, location data indicative of the location at which the parameter value was acquired (e.g. geographical coordinates, WiFi Internet Protocol (IP) address, etc.), a sensor type, information enabling identification of a specific sensor with which the parameter value was acquired, Inertial Navigation System (INS) and/or pressure sensor and/or room humidity and/or room temperature and/or patient orientation and/or room ambient noise level readings acquired during acquisition of the parameter value.

Central system 130 can further comprise management system 132 configured to forward medical data acquired by the medical data acquisition device 104 (whether in a raw form, or any processed version of the raw data acquired by the medical data acquisition device 104) and relating to a patient 103, and optionally other patient-related data relating to the patient 103, to a selected medical practitioner workstation 122 (for example an available medical practitioner workstation 122 or medical practitioner workstation 122 with the shortest queue, e.g. in case where no medical practitioner, out of a plurality of medical practitioners, is currently available). It is to be noted that when providing a central system 130, there may be more than one medical practitioner location 120 and more than one medical practitioner 124 as central system 130 can allow the distributed approach in which data (e.g. medical data and/or other patient-related) can be received by the central system 130 from multiple patient locations 100 and transferred by it to multiple medical practitioner locations 120.

Having described the various components in the patient location 100, in the medical practitioner location 120 and the central system 130, attention is drawn to two exemplary modes of operation of the medical data acquisition device 104: an on-line mode and an off-line mode.

In an on-line mode, a medical examination of the patient 103 is conducted while the medical practitioner 124 is actively involved in the process. In such operation mode, the medical practitioner 124 can be provided with a video or a sequence of images, based on which the medical practitioner 124 provides the user 102 with instructions for positioning the medical data acquisition device 104 with respect to the patient's 103 body. In addition, the medical practitioner 124 can provide the user 102 with instructions for performing a current medical examination (other than positioning instructions) and/or with instructions for performing other medical examinations as part of the medical examination flow. In some cases, the instructions can be audible instructions, acquired by a microphone on the medical practitioner location (e.g. a microphone connected to the medical practitioner workstation 122), and provided to the user 102 via a speaker in the patient location 100 (e.g. a speaker of the medical data acquisition device 104, a speaker of the patient workstation 114, or any other speaker which provides sounds that the user 102 can hear). Additionally, or alternatively to the audible instructions, the instructions can be video instructions provided via a display in the patient location 100 (e.g. a display of the medical data acquisition device 104, a display of the patient workstation 114, or any other display visible to the user 102).

The video that is provided to the medical practitioner 124 can be acquired by a camera comprised within the medical data acquisition device 104 (e.g. one of the sensors 106 can be a camera used for this purpose), and in such case, the medical practitioner 124 can view the part of the patient's body to which the camera is aimed. In additional, or alternative cases, the video can be acquired by an external camera 110 external to the medical data acquisition device 104, and in such cases, the medical practitioner 124 can view the patient 103 and the medical data acquisition device 104 in the same frame. In any case, based on the camera's view, the medical practitioner 124 can provide the user 102 with maneuvering instructions for navigating the medical data acquisition device 104 to a desired spatial disposition with respect to the patient's 103 body. In some cases, the video can be accompanied by a sound recording acquired using a microphone located at the patient location 100 (e.g. a microphone of the medical data acquisition device 104, a microphone of the patient workstation 114, or any other microphone that can acquire a sound recording of sounds at the patient location 100).

Upon arrival of the medical data acquisition device 104 to the desired spatial disposition (from which the medical data can be acquired) with respect to the patient's 103 body, the medical practitioner 124 can instruct the user 102 to acquire the medical data (and optionally also instruct the user 102 on how to acquire the medical data), or it can operate the sensors 106 himself to acquire the medical data. In some cases, the medical practitioner 124 can also remotely control various parameters of the sensors 106, e.g. through medical practitioner workstation 122.

It is noted that medical data acquisition device 104 can be located outside the body of the patient 103 when acquiring the medical data. Nevertheless, in some cases some parts of medical data acquisition device 104 may enter the body of the patient 103 (e.g. a needle penetrating the skin and/or a blood vessel, a sensor entering a body orifice such as the ear or the mouth, a swab entering a body orifice for collecting a sample, and so on). Even in such cases, the greater part of medical data acquisition device 104 can be located outside the body at the time of measurement and/or data/sample acquisition.

The medical data acquired by the medical data acquisition device 104 can be transmitted to the medical practitioner workstation 122 (directly, or through the patient workstation 114 and/or through the central system 130 where it can be stored in the medical check repository 134 in association with the patient 103 from which the medical data was acquired), where it can be stored in medical practitioner data repository 123 in association with the patient 103 from which the medical data was acquired.

The medical practitioner 124 (e.g. a doctor, a nurse, a medic, etc., including any other person with the know-how and skill to acquire and/or analyze medical data), located at medical practitioner location 120, can review the acquired medical data, for example using medical practitioner workstation 122. It is to be noted that patient workstation 114, medical practitioner workstation 122 and central system 130 can include a display (e.g. LCD screen), and a keyboard or any other suitable input/output devices.

In some cases, medical practitioner 124 can provide feedback data (e.g. by transmitting corresponding instructions to patient workstation 114 and/or to medical data acquisition device 104) to user 102, such as a diagnosis, one or more prescriptions, or instructions to perform one or more additional medical examinations. Alternatively, or additionally, medical practitioner 124 can transmit feedback data to central system 130, which, in turn, can optionally transmit the feedback data to patient workstation 114 and/or to the medical data acquisition device 104 (e.g. via the communication network 116).

In some cases, the medical data acquisition device 104 and/or the patient workstation 114 can be configured to provide the user 102 with an indication of a quality of a signal acquired by the sensors. In such cases, the medical data acquisition device 104 and/or the patient workstation 114 can be configured to determine the signal quality and display an appropriate indication on a display visible by the user 102 (e.g. a display of the medical data acquisition device 104 and/or a display of the patient workstation 114). In some cases, upon the signal quality not meeting pre-defined thresholds, the medical data acquisition device 104 and/or the patient workstation 114 can be configured to provide the user 102 with instructions for improving the acquired signal quality (e.g. instructions to reposition the medical data acquisition device 104, instructions to reduce ambient noise, etc.).

In an off-line mode, a medical examination of the patient 103 is conducted while no medical practitioner 124 is actively involved in the process. In such operation mode, the medical data acquisition device 104 can provide the user 102 with audio and/or video navigation instructions for navigating the medical data acquisition device 104 to a desired spatial disposition with respect to the patient's 103 body. The navigation instructions can be determined by the medical data acquisition device 104 and/or by the patient workstation 114 using information obtained from an Inertial Navigation System (INS), that can optionally be part of the sensors 106, and/or using matching of reference points within reference images and images acquired by a camera comprised within the medical data acquisition device 104 and/or by external camera 110. The navigation instructions can be provided via a speaker and/or a display of the medical data acquisition device 104 and/or of the patient workstation 114 and/or of any other device located near the user 102 in a manner that enables a user to hear and/or see the navigation instructions.

Upon arrival of the medical data acquisition device 104 to the desired spatial disposition (from which the medical data can be acquired) with respect to the patient's 103 body, the user 102 can operate the medical data acquisition device 104 to acquire medical data, or alternatively, the medical data acquisition device 104 can acquire the medical data automatically.

In some cases, the medical data acquisition device 104 and/or the patient workstation 114 can be configured to provide the user 102 with an indication of a quality of a signal acquired by the sensors. In such cases, the medical data acquisition device 104 and/or the patient workstation 114 can be configured to determine the signal quality and display an appropriate indication on a display visible by the user 102 (e.g. a display of the medical data acquisition device 104 and/or a display of the patient workstation 114). In some cases, upon the signal quality not meeting pre-defined thresholds, the medical data acquisition device 104 and/or the patient workstation 114 can be configured to provide the user 102 with instructions for improving the acquired signal quality (e.g. instructions to reposition the medical data acquisition device 104, instructions to reduce ambient noise, etc.).

It is noted that medical data acquisition device 104 can be located outside the body of the patient when acquiring the medical data. Nevertheless, in some cases some parts of medical data acquisition device 104 may enter the body of the patient (e.g. a needle penetrating the skin and/or a blood vessel, a sensor entering a body orifice such as the ear or the mouth, a swab entering a body orifice for collecting a sample, and so on). Even in such cases, the greater part of medical data acquisition device 104 can be located outside the body at the time of measurement and/or data/sample acquisition.

In some cases, the medical data that is acquired by the sensors can be received in two (or more) different channels, while the medical data sent via each channel has a different quality.

For example, a first channel can include the medical data in a first quality, that is sufficient for enabling the medical practitioner 124 to provide the user 102 with navigation and/or positioning instructions for navigating the medical data acquisition device 104 to a desired spatial disposition with respect to the patient's 103 body, and/or for placing the medical data acquisition device 104 at a desired placement (e.g. a desired pressure level) with respect to the patient's 103 body. A second channel can include the medical data in a second quality, better/higher than the first quality, that is more likely to enable the medical practitioner 124 to accurately diagnose the patient 103 (e.g. enabling the medical practitioner 124 to determine if noises in readings which include a recording of the lungs are related to a medical condition of the patient 103 or simply reading noise, or to determine if a suspicious area on the patient's 103 skin is dirt or a mole that requires analysis).

The medical data acquired by the medical data acquisition device 104 can be transmitted to a medical practitioner workstation 122 (directly, or through the patient workstation 114 and/or through the central system 130 where it can be stored in the medical check repository 134 in association with the patient 130 from which the medical data was acquired), where it can be stored in medical practitioner data repository 123 in association with the patient 130 from which the medical data was acquired.

The medical practitioner 124 (e.g. a doctor, a medic, etc., including any other entity (human or computerized) with the know-how and skill to acquire and/or analyze medical data), located at medical practitioner location 120, can review the acquired medical data, for example using a display and/or a speaker and/or any other suitable output device of the medical practitioner workstation 122. It is to be noted that patient workstation 114, medical practitioner workstation 122 and central system 130 can include a display (e.g. LCD screen), and a keyboard or any other suitable input/output devices.

In some cases, medical practitioner 124 can provide feedback data (e.g. by transmitting corresponding instructions to patient workstation 114 and/or to medical data acquisition device 104) to user 102, such as a diagnosis, one or more prescriptions, or instructions to perform one or more additional medical examinations. Alternatively, or additionally, medical practitioner 124 can transmit feedback data to central system 130, which, in turn, can optionally transmit the feedback data to patient workstation 114 (e.g. via the communication network 116). As indicated herein, the feedback data can be provided to the user 102 via an output device (e.g. a display, a speaker, etc.) of the medical data acquisition device 104 and/or of the patient workstation, or of any other device that is capable of providing the respective output to the user 102.

It is to be noted that in some cases, the medical practitioner data repository 123 and/or the data repository 107, and or the medical check repository 134 and/or the patient & check plan repository 136 can be the same single data repository, whether distributed or not, that is accessible by all relevant entities.

Furthermore, at least one of the sensors 106 can be a camera which is configured to capture images that include at least a part of the swab upon the swab being connected to the medical data acquisition device 104 via the mechanical connection 108. The mechanical connection 108 can be designed so that upon connection of the swab thereto the tip of the swab is located within camera's field of view thereby enabling the camera to capture images that include at least a far end of the swab (i.e. the tip of the swab).

In some cases, the camera can be an external camera 110 (i.e. external to medical data acquisition device 104 and movable irrespective thereto) that can capture images that include at least a part of the swab upon the swab being connected to the medical data acquisition device 104 via the mechanical connection 108 (e.g. during a nasopharyngeal sample collection as further detailed hereinbelow). In such cases, the external camera 110 can communicate with medical data acquisition device 104 and/or patient workstation 114 and/or with medical practitioner workstation 122 and/or with central system 130, through communication network 116 (e.g. the Internet), via wired or wireless communication, to transmit said captured images.

Attention is now drawn to FIG. 2, a flowchart illustrating one example of a sequence of operations carried out for performing a process 200 for collecting a sample, in accordance with the presently disclosed subject matter.

For this purpose, the medical data acquisition device 104, utilizing the processing circuitry 105, can be configured to operate a camera (being one of the sensors 106 or an external camera 110, external to the medical data acquisition device 104 and movable irrespective thereto) to acquire at least one image during the process for collecting a sample by a tip of a swab, making contact with a required contact area on, or within, a body of a patient (block 210).

As indicated herein, in some cases, the camera can be comprised within the medical data acquisition device 104, and the medical data acquisition device 104 further comprises a mechanical connection 108 for connecting the swab so that images captured by the camera include at least a part of the swab when the swab is connected to the medical data acquisition device 104 via the mechanical connection 108. In more specific cases, the medical data acquisition device 104 can be a smartphone and the mechanical connection 108 can be an add-on connectable to the smartphone for enabling positioning of the swab so that images captured by the camera include at least part of the swab when the swab is connected to the medical data acquisition device 104 via the mechanical connection 108.

In other cases, the medical data acquisition device 104 can comprise a mechanical connection 108 for connecting the swab and the camera can be external to the medical data acquisition device 104 (e.g. external camera 110) and movable irrespective of the swab, even when the swab is connected to the medical data acquisition device 104 via the mechanical connection 108.

In still other cases, the swab is not connected to the medical data acquisition device 104 whatsoever, and the camera, whether comprised within the medical data acquisition device 104 or external to the medical data acquisition device 104 (e.g. external camera 110) and movable irrespective thereto. In such cases, the swab can be held, for example, by the user 102 in one hand, while the camera is stationary or held by the user's 102 other hand. It is to be noted in this respect that in some cases the user 102 can be the patient 103 performing sample collection by himself.

The process 200 can involve the user 102 placing the swab in a required position and/or orientation, on or within the body of the patient 103, wherein the tip of the swab is making contact with the required contact area. In some cases, the swab can be inserted into a nasopharyngeal and/or oropharyngeal cavity of the patient 103 for diagnosing upper respiratory tract infections, such as but not limited to, Coronavirus disease (COVID-19), Whooping cough, Streptococcal pharyngitis, etc. In other cases, the swab can be placed on the body of the patient 103 at a required position and/or orientation (e.g. eye or skin), e.g. for diagnosing skin lesions (e.g. Herpes (HSV)), etc.

The required position and/or orientation of the swab can be determined by the medical data acquisition device 104 based on the medical condition to be diagnosed. For example, for diagnosing a Group A streptococcus a throat culture should be tested. In such cases the required contact area of the tip of the swab within the body of the patient 103 can be the back of the throat (e.g. white dots or patches in the back of the throat, around the tonsils, red areas or sores, inflammation or pus and so on). In another example, to test for COVID-19, nasopharyngeal sample should be collected by the tip of the swab. In such cases the required contact area of the tip of the swab within the body of the patient 103 can be nasopharynx that is located inside a nasal cavity.

In some cases, the processing circuitry 105 can be further configured to identify, within the at least one image, the required contact area, or a cavity leading to the required contact area. For example, in order to test for COVID-19, nasopharyngeal sample should be collected by the tip of the swab. In such cases, the required contact area is located inside a nasal cavity. The processing circuitry 105 can identify, within the at least one image acquired during the process for collecting the sample by the tip of the swab, the cavity leading to the required contact area, in this case the nasal vestibule.

In some cases, the required contact area can be determined by an operator of the medical data acquisition device 104. The operator can be the user 102 or the patient 103, as detailed hereinabove with respect to FIG. 1. Alternatively, the operator can be a medical practitioner that is specifically trained to acquire medical data from the patient's 103 body (e.g. a doctor, clinician, registered nurse, etc.) and optionally to diagnose a medical condition of the patient 103 based on medical data acquired from the patient's 103 body.

In some cases, the operator can be located at a remote location, remote from the patient 103. For example, the operator can be the medical practitioner 124 that is located at the medical practitioner location 120, remote from the patient location 100 (and in such cases, the medical practitioner 124 can identify the required contact area on an image that is displayed thereto—be it an image acquired by the one of the sensors 106 or by the external camera 110).

Medical data acquisition device 104 can be further configured to analyze the image to determine one or more parameters, the parameters including at least one of (a) a position of the swab with respect to the required contact area, or (b) an orientation of the swab with respect to the required contact area (block 220).

During the process for collecting the sample from patient's 103 body it is essential to determine the position and/or orientation of the swab or portion thereof with respect to the required contact area. This can be achieved by analyzing the at least one acquired image in order to determine if the tip of the swab is making contact with the required contact area or located at required contact area on or within the body of the patient 103. For this purpose, one or more parameters can be determined by the medical data acquisition device 104. One of the parameters can be position of the swab with respect to the required contact area. For example, for collecting nasopharyngeal sample from patient's 103 body the position of the swab with respect to the required contact area (e.g. nostril) can be determined by the medical data acquisition device 104. Another parameter can be orientation of the swab with respect to the required contact area. For example, for collecting nasopharyngeal sample from patient's 103 body the orientation of the swab with respect to the required contact area (e.g. nostril) can be determined by the medical data acquisition device 104.

In some cases, the swab can include one or more scale marks and/or graduation marks thereon, that can enable determining a penetration depth of the swab into a body cavity of the patient 103 (e.g. a nostril of the patient 103). Additionally, or alternatively, the swab can include one or more rotation determination marks thereon, that enable determination of a rotation of the swab around an axis of rotation thereof. In some cases, the rotation determination marks can include two non-parallel lines painted (or otherwise applied) on the swab. In such cases, a change in the distance between the two lines is indicative of rotation of the swab when such change is viewed from a fixed point of view with respect to the swab. Attention is drawn in this respect to FIG. 10, showing an example of a swab 1000 with an exemplary graduation mark 1010 enabling determination of a penetration depth of the swab into a cavity, and a rotation determination mark 1020 enabling determining rotation of the swab.

Medical data acquisition device 104 can be further configured to perform at least one action based on comparison of the parameters with a positioning criterion (block 230).

Each process for collecting a sample from patient's 103 body can be associated with a corresponding positioning criterion wherein one or more parameters can be compared thereto and thereby determining if the tip of the swab is making contact with the required contact area or located at required contact area on or within the body of the patient 103. The positioning criterion indicates the required position and/or orientation of the swab, on or within patient's 103 body, for collecting an adequate sample and may vary based on the particular diagnostic test type to be taken. For example, to collect a sample to test for Group A streptococcus the positioning criterion can be white dots or patches in the back of the throat whereas to test for Herpes (HSV) the positioning criterion can be eye or skin.

In some cases, more than one positioning criterion may be associated with a specific process for collecting a sample from patient's 103 body. For example, in order to test for COVID-19, nasopharyngeal sample should be collected by the tip of the swab from both sides of the nose in order to ensure that enough material is collected. Therefore, the positioning criterion for one side of the nose can be different from the positioning criterion for the other side thereof.

In some cases, upon the comparison of the one or more parameters with the positioning criterion indicating that the swab is not at a desired spatial position with respect to the required contact area, the medical data acquisition device 104 can be further configured to perform at least one of the following actions: alerting the user 102 of the medical data acquisition device 104 or marking the sample as invalid. For example, if during the process for collecting a sample from patient's 103 body to test for Group A streptococcus the tip of the swab is not located at or near white dots in the back of the throat of the patient 103 the medical data acquisition device 104 can alert the user 102 of incorrect placement of the tip of the swab, instruct the user 102 to replace the swab with a new swab, mark the sample as invalid, etc.

In other cases, upon the comparison of the one or more parameters with the positioning criterion indicating that the swab is at a desired spatial position with respect to the required contact area, the medical data acquisition device 104 can be further configured to perform at least one of the following actions: providing the user 102 of the medical data acquisition device 104 with a successful positioning indication or marking the sample as valid. For example, if during the process for collecting a sample from patient's 103 body to test for Group A streptococcus the tip of the swab is located at or near white dots in the back of the throat of the patient 103 the medical data acquisition device 104 can provide the user 102 with a successful positioning indication of the tip of the swab, instruct the user 102 to proceed with collecting the sample, mark the sample as valid, etc.

In some cases, the required contact area can be within a cavity of the body the patient 103 (e.g. nostril). In such cases, the positioning criterion may depend on a length of an inserted part of the swab inserted into the cavity. For example, in order to collect a sample to test for COVID-19, the swab should be inserted through the nares of the patient 103 so that it reaches depth equal to distance from nostrils to outer opening of the ear. The medical data acquisition device 104 is configured to assess the length of the portion of the swab inserted into the nostril (e.g. by image analysis) and thereby determine if it meets the positioning criterion parameters, i.e. the tip of the swab is located at the required contact area within the nostril of the patient 103 (e.g. is in contact with the nasopharynx). It is to be noted, as indicated herein, that in some cases, for this purpose, the swab can include one or more scale marks and/or graduation marks thereon, that can enable determining a penetration depth of the swab into a body cavity of the patient 103 (as illustrated, for example, in FIG. 10).

In some cases, the length of the inserted part of the swab inserted into the cavity depends on parameters of the patient 103, such as age, height, gender, a structure of the cavity etc. For example, some studies show that after age of 16 nasal cavities are bigger in men than in women, also that intranasal spaces can change with age (e.g. children vs adults). Therefore, the length of the inserted part of the swab inserted into a nose of a child will differ from the length of the inserted part of the swab inserted into a nose of an adult.

In some cases, the processing circuitry 105 can be further configured to provide the user 102 of the medical data acquisition device 104 with a successful sampling indication upon meeting a sampling criterion. The sampling criterion can depend on the particular diagnostic test type to be taken. For example, during the process for collecting nasopharyngeal sample from patient's 103 body to test for COVID-19, the tip of the swab should be gently rubbed and rolled against the nasopharynx and left in place for several seconds to absorb secretions thereof. In such cases, the sampling criterion can be time elapsed above predetermined threshold, swivel of the swab above predetermined number of times (noting that in some cases, this can be determined utilizing rotation determination marks (e.g. rotation determination mark 1020 shown in FIG. 10) painted (or otherwise applied) on the swab in cases where the swab is not connected to the medical data acquisition device 104 via a mechanical connection 108, or using accelerometers or other orientation determination sensors of the medical data acquisition device 104 when the swab is connected to the medical data acquisition device 104), and so on. For example, if the swab was rotated at least 15 times while located at the required contact area within patient's 103 nose, then the medical data acquisition device 104 can provide the user 102 with a successful sampling indication. In another example, during the process for collecting oropharyngeal sample from patient's 103 body to test for Group A streptococcus, the tip of the swab should be moved in a sweeping motion to swab the posterior pharyngeal wall and tonsillar pillars. In such cases, the sampling criterion can be for example pressure applied on the swab over a predetermined duration of time. The pressure can be measured by at least one of the sensors 106 that can be a pressure sensor which is configured to measure pressure applied on the swab upon the swab being connected to the medical data acquisition device 104 via the mechanical connection. Alternatively, or additionally, the pressure can be measured by analyzing and comparing images acquired during the process for collecting the sample from patient's 103 body wherein at least one image was acquired upon arrival the tip of the swab to the desired spatial position with respect to the required contact area and at least one image was acquired upon expiration of the predetermined duration of time. When comparing said images the shaft of the swab can look bent and the degree of curvature thereof can indicate the amount of pressure applied thereon.

In some cases, the sampling criterion can be based on a predetermined protocol. The protocol can comprise information regarding various sampling procedures (e.g. pressure applied on the swab over a predetermined duration of time, required motion of the swab at the required contact area, etc.), each associated with the particular diagnostic test type to be taken. Said protocol can be occasionally replaced or updated with new or additional sampling procedures (e.g. the sampling procedures can correspond to new medical developments or can be based on a medical data retrieved from other medical data acquisition devices). In some cases, said changes to the protocol can be performed remotely (e.g. by downloading an update to the medical data acquisition device 104 via the internet).

In some cases, before initiating the process for collecting a sample from patient's 103 body, the user 102 can input the medical data acquisition device 104 the particular diagnostic test type to be taken. As a response the medical data acquisition device 104 can be configured to provide guiding instructions to the user 102, such as but not limited to, an explanation on how to attach the swab to the swab mechanical connection 108, a video illustrating how to collect the required sample, navigation instructions indicating the required contact area and how to arrive thereto, adequate sample collection guide, instructions for placing the swab after collecting the sample in a correct receptacle (e.g. test tube) and so on.

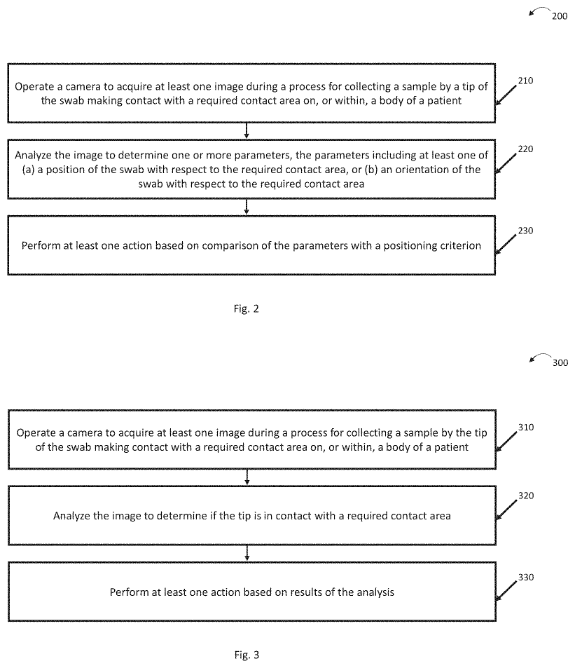

In some cases, the medical data acquisition device 104 can be configured to perform process 300 depicted in FIG. 3. In such cases, the camera can be configured to capture images that include at least a tip of the swab upon the swab being connected to the medical data acquisition device 104 via the mechanical connection.

The processing circuitry 105, can be configured to operate the camera (being one of the sensors 106 or an external camera 110, external to the medical data acquisition device 104 and movable irrespective thereto) to acquire at least one image during the process for collecting a sample by a tip of the swab making contact with a required contact area on, or within, a body of a patient (block 310).

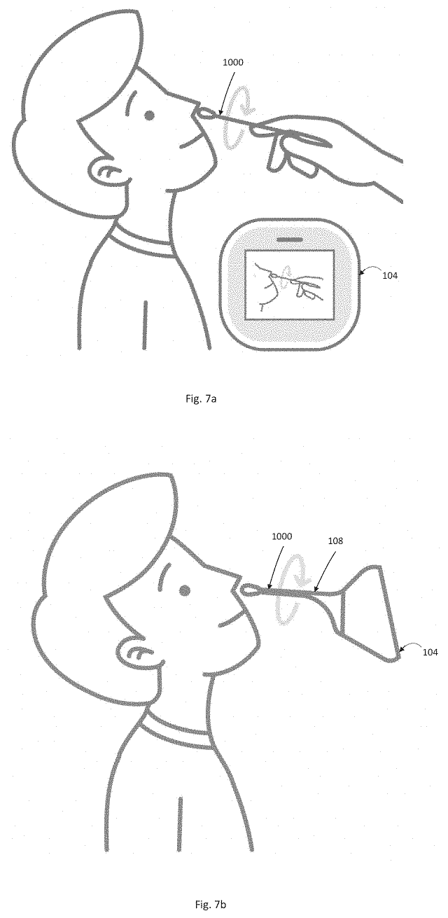

As indicated herein, in some cases, the camera can be comprised within the medical data acquisition device 104, and the medical data acquisition device 104 further comprises a mechanical connection 108 for connecting the swab so that images captured by the camera include at least a part of the swab when the swab is connected to the medical data acquisition device 104 via the mechanical connection 108. Attention is drawn in this respect, for purpose of illustration, to FIG. 7b, showing a swab 1000 connected to a medical data acquisition device 104 via a mechanical connection 108.

In more specific cases, the medical data acquisition device 104 can be a smartphone and the mechanical connection 108 can be an add-on connectable to the smartphone for enabling positioning of the swab so that images captured by the camera include at least part of the swab when the swab is connected to the medical data acquisition device 104 via the mechanical connection 108.

In other cases, the medical data acquisition device 104 can comprise a mechanical connection 108 for connecting the swab and the camera can be external to the medical data acquisition device 104 (e.g. external camera 110) and movable irrespective of the swab, even when the swab is connected to the medical data acquisition device 104 via the mechanical connection 108.

In still other cases, the swab is not connected to the medical data acquisition device 104 whatsoever, and the camera, whether comprised within the medical data acquisition device 104 or external to the medical data acquisition device 104 (e.g. external camera 110) and movable irrespective thereto. In such cases, the swab can be held, for example, by the user 102 in one hand, while the camera is stationary or held by the user's 102 other hand. It is to be noted in this respect that in some cases the user 102 can be the patient 103 performing sample collection by himself. Attention is drawn in this respect to FIG. 7a showing a swab 1000 that is not connected to a medical data acquisition device 104, but rather held by one hand of the user 102. In the illustration provided in FIG. 7a, the camera is comprised within the medical data acquisition device 104, however, as indicated herein, this is by no means limiting, and the camera can be external to the medical data acquisition device 104, mutatis mutandis.

For example, to collect a sample to test for Herpes (HSV), the camera can acquire at least one image during the process for collecting the sample by the tip of the swab wherein the tip of the swab is located within camera's field of view (FOV). That is, the camera can capture the tip of the swab making contact with the required contact area on, or within, the body of the patient 103 (e.g. eye or skin).

Medical data acquisition device 104 can be further configured to analyze the at least one image to determine if the tip is in contact with a required contact area (block 320).

During the process for collecting the sample from patient's 103 body it is essential to determine the location of the tip of the swab with respect to the required contact area. This can be achieved by analyzing the at least one acquired image in order to determine if the tip of the swab is making contact with the required contact area or located at required contact area on or within the body of the patient 103.

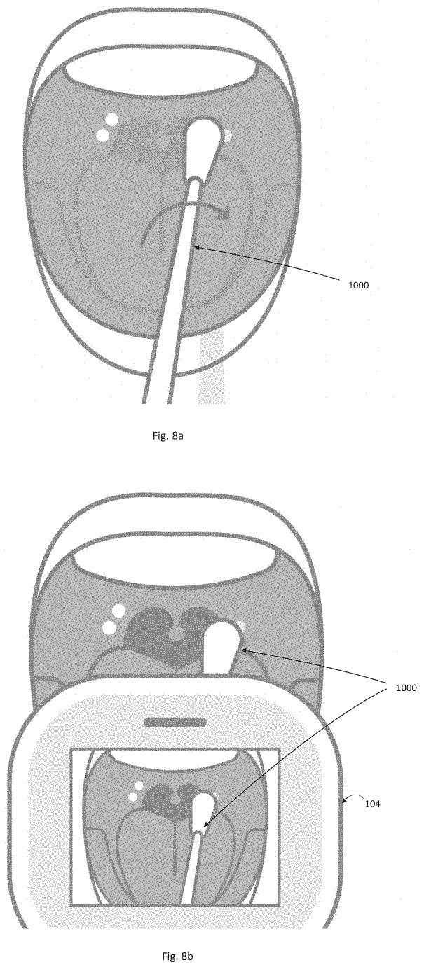

For example, for collecting oropharyngeal sample from patient's 103 body the required contact area can be white dots or patches in the back of the throat. The medical data acquisition device 104 is configured to analyze the at least one image acquired during the process for collecting the sample from patient's 103 body to determine if the tip of the swab is in contact with said required contact area. Attention is drawn in this respect to FIG. 8a, showing a swab 1000 inserted into the back of the throat of the patient 103, in an attempt to collect a sample from white dots within the patient's 103 throat. Looking at FIG. 8b, there is shown a medical data acquisition device 104 used during the sample collection process. The medical data acquisition device 104 captures images including the swab 1000 during the sample collection process and such images are utilized to determine if the tip of the swab 1000 is in contact with a required contact area (e.g. the white dots within the patient's 103 throat).

Medical data acquisition device 104 can be further configured to perform at least one action based on results of the analysis (block 330).

Upon determination of the medical data acquisition device 104 if the tip of the swab is in contact with a required contact area (e.g. by analyzing the at least one acquired image) the system can perform at least one action as further detailed hereinbelow.

In some cases, upon the determination indicating that the tip of the swab is not in contact with the required contact area the medical data acquisition device 104 can perform at least one of the following actions: alerting the user 102 of the medical data acquisition device 104 or marking the sample as invalid. For example, if during the process for collecting a sample from patient's 103 body to test for Group A streptococcus the tip of the swab is not located at or near white dots in the back of the throat of the patient 103 the medical data acquisition device 104 can alert the user 102 of incorrect placement of the tip of the swab, instruct the user 102 to replace the swab with a new swab, mark the sample as invalid, etc.

In other cases, upon the determination indicating that the tip of the swab is in contact with the required contact area the medical data acquisition device 104 can perform at least one of the following actions: providing the user 102 of the medical data acquisition device 104 with a successful positioning indication or marking the sample as valid. For example, if during the process for collecting a sample from patient's 103 body to test for Group A streptococcus the tip of the swab is located at or near white dots in the back of the throat of the patient 103 the medical data acquisition device 104 can provide the user 102 with a successful positioning indication of the tip of the swab, instruct the user 102 to proceed with collecting the sample, mark the sample as valid, etc.

In some cases, the processing circuitry 105 can be further configured to analyze the image, acquired during the process for collecting a sample from patient's 103 body, to determine the required contact area. For example, to collect a sample to test for Group A streptococcus the required contact area can be white dots or patches in the back of the throat.

In some cases, the required contact area can be determined by an operator of the medical data acquisition device 104. The operator can be the user 102 or the patient 103, as detailed hereinabove with respect to FIG. 1. Alternatively, the operator can be a medical practitioner that is specifically trained to acquire medical data from the patient's 103 body (e.g. a doctor, clinician, registered nurse, etc.) and optionally to diagnose a medical condition of the patient 103 based on medical data acquired from the patient's 103 body.

In some cases, the operator can be located at a remote location, remote from the patient 103. For example, the operator can be the medical practitioner 124 that is located at the medical practitioner location 120, remote from the patient location 100.

In some cases, the medical data acquisition device 104 can be configured to acquire at least one image during the process for collecting a sample by a tip of a swab in order to validate that the tip of the swab is making contact with a required contact area on, or within, a body of a patient, as detailed hereinabove with respect to FIGS. 2-3. This way, when the user 102 of the medical data acquisition device 104 believes that the tip of the swab is placed properly (i.e. in a required location on, or within, a body of a patient) the medical data acquisition device 104 can validate swab's placement (e.g. by analyzing the at least one acquired image) before collecting the sample.

In other cases, the medical data acquisition device 104 can be configured to acquire a sequence of images during the process for collecting a sample by the tip of the swab, inter alia, to assist the user 102 with placing the tip of the swab in a correct location by providing him navigation instructions based on the acquired sequence of images, as further detailed hereinbelow with respect to FIG. 4.

In some cases, the medical data acquisition device 104 can be configured to perform a navigation process 400 depicted in FIG. 4.

For this purpose, the processing circuitry 105 can be configured to operate a camera (being one of the sensors 106 or an external camera 110, external to the medical data acquisition device 104 and movable irrespective thereto) to acquire a sequence of images during the process for collecting the sample from the body of the patient 103 (block 410).

The sequence of images can be acquired during user's 102 movement of the medical data acquisition device 104, which results in respective movement of the tip of the swab (upon connection thereof to the medical data acquisition device 104 via the mechanical connection 108), in order to track spatial movement of the tip of the swab towards the required contact area on or within the body of the patient 103 which is based on the particular diagnostic test type to be taken. For example, if a nasopharyngeal sample is to be taken from within the body of the patient 103 then the camera of the medical data acquisition device 104 can track the spatial movement of the tip of the swab towards patient's 103 nostril.

Medical data acquisition device 104 can be further configured to analyze the sequence of images to determine one or more navigation parameters, the navigation parameters including at least one of: (a) a navigation position of the swab with respect to the required contact area, or (b) a navigation orientation of the swab with respect to the required contact area (block 420).

In some cases, during the process for collecting the sample from patient's 103 body, the medical data acquisition device 104 can analyze the sequence of images to determine if the tip of the swab is making contact with the required contact area or located at required contact area on or within the body of the patient 103. For this purpose, one or more navigation parameters can be determined by the medical data acquisition device 104. One of the navigation parameters can be navigation position of the swab with respect to the required contact area. For example, for collecting oropharyngeal sample from patient's 103 body the navigation position of the tip of the swab with respect to the required contact area (e.g. white dots or patches in the back of the throat) can be determined by the medical data acquisition device 104. Another parameter can be navigation orientation of the swab with respect to the required contact area. For example, for collecting nasopharyngeal sample from patient's 103 body the navigation orientation of the swab with respect to the required contact area (e.g. nostril) can be determined by the medical data acquisition device 104.

Medical data acquisition device 104 can be further configured to provide the user 102 with navigation instructions, based on the navigation parameters, for navigating the tip of the swab to contact the required contact area (block 430).

In some cases, the navigation parameters can indicate that the movement of the tip of the swab is not towards the required contact area on or within the body of the patient 103. In such cases, the medical data acquisition device 104 can provide the user 102 with navigation instructions towards the required contact area that can be based on the navigation parameters. For example, if a nasopharyngeal sample is to be taken and the navigation parameters indicate that the tip of the swab is moved towards the mouth of the patient 103, the medical data acquisition device 104 can provide the user 102 with navigation instructions from tip's current location towards the patient's 103 nostril.

In other cases, the navigation parameters can indicate that the movement of the tip of the swab towards the required contact area, on or within the body of the patient 103, was interrupted before arriving to the required contact area. In such cases, the medical data acquisition device 104 can provide the user 102 with navigation instructions towards the required contact area, based on the navigation parameters. For example, if a nasopharyngeal sample is to be taken and the navigation parameters indicate that the tip of the swab arrived to patient's 103 nostril but was not advanced therein, the medical data acquisition device 104 can provide the user 102 with navigation instructions for inserting the tip of the swab inside patient's 103 nostril.

In some cases, the navigation instructions can be voice instructions generated by speakers (of the medical data acquisition device 104 or patient workstation 114), visual instructions (e.g. arrow/text/color/icon indicators) provided on a display (of the medical data acquisition device 104 or patient workstation 114), vibrations of the medical data acquisition device 104 generated to draw user's attention thereto, etc.

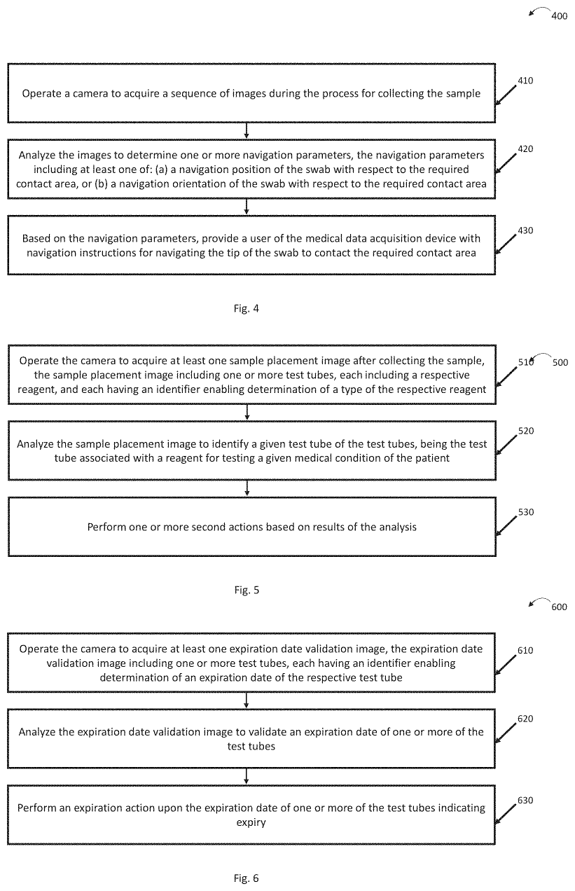

In some cases, the medical data acquisition device 104 can be configured to perform a sample placement verification process 500, e.g. as depicted in FIG. 5.

For this purpose, the processing circuitry 105 can be configured to operate a camera (being one of the sensors 106 or an external camera 110, external to the medical data acquisition device 104 and movable irrespective thereto) to acquire at least one sample placement image after collecting the sample, the sample placement image including one or more receptacles (e.g. test tubes, glasses, cassettes, cuvettes or any other type of receptacle into which the swab can be inserted), each including a respective reagent, and each having an identifier enabling determination of a type of the respective reagent (block 510).

Upon obtaining a successful sample from the required contact area, on or within the body of the patient 103, the swab can be placed inside an appropriate receptacle. In some cases, the receptacle can be part of a kit including one or more receptacles used to collect and hold samples obtained from the body of the patient 103. Said receptacles can be identical or have different chemical additives comprised therein (e.g. reagent). It is to be noted that in some cases, for example and without limitation, the chemical additives may be transparent in the visible spectrum and therefore an appropriate identifier is needed for enabling determination of a type of the respective reagent comprised within each receptacle. In case of different receptacles, each receptacle can have a unique identifier enabling determination of a type of the respective chemical additive(s) comprised therein. The unique identifier can be for example and without limitation a rubber stopper having a unique color indicating the additive, a barcode, a snap-top seal, a unique shape and/or color(s) of the receptacle, a unique shape and/or color(s) of one or more portions of the receptacle (for example a base portion of one or more receptacles may have a unique distinctive color), a label located on the receptacle and/or on a corresponding lid thereof, a Radio-frequency identification (RFID) and/or any combination(s) thereof and/or any other identifier that may be suitable for enabling unique identification in accordance with the presently disclosed subject matter.

In order to provide the patient 103 with an accurate medical diagnosis it is necessary to ensure that the sample, collected during the process for collecting sample from patient's 103 body utilizing the tip of the swab, is placed inside an appropriate receptacle having a correct chemical additive(s) comprised therein (i.e. reagent corresponding to the particular diagnostic test type that was taken).

In some cases, the processing circuitry 105 can be further configured to operate the camera to acquire at least one sample placement image after collecting the sample. The least one sample placement image can include an image of the swab used for collecting the sample from patient's 103 body placed within a receptacle.

The processing circuitry 105 can be further configured to analyze the sample placement image to identify a given receptacle of the receptacles, being the receptacle associated with a reagent for testing a given medical condition of the patient (block 520).

In some cases, the processing circuitry 105 can analyze the sample placement image in order to verify that the swab used for collecting the sample from patient's 103 body was placed within a correct receptacle. The correct receptacle is a receptacle that includes a reagent that corresponds to the particular diagnostic test type that was taken. The identification of the correct receptacle can be based on the unique identifier of the receptacle. For example, a receptacle associated with Group A streptococcus test can have a yellow lid while a receptacle associated with COVID-19 test can have a red lid. If a process for collecting a sample for testing COVID-19 was performed, the processing circuitry 105 can identify, by analyzing the sample placement image, if the swab was placed within the receptacle with the red lid.

It is to be noted that in some cases, the user 102 may be required to add one or more chemical compounds into the correct receptacle (where they are optionally mixed with existing chemical compounds that already exist within the correct receptacle), and in such cases, the processing circuitry 105 can be further configured to verify that the correct chemical compounds, and optionally the correct amount of correct chemical compounds (e.g. a correct number of drops), are added to the correct receptacle in which the swab with the sample obtained from the patient's 103 body is placed (whether before, or after, the swab is placed inside the receptacle). For this purpose, the processing circuitry 105 can operate the camera to acquire chemical compound addition images, analyze the images to identify the compound added to the correct receptacle (e.g. using an identifier of the chemical compound that can be, for example, printed on a receptacle of the chemical compound added to the receptacle into which the swab with the sample obtained from the patient's 103 body is to be inserted), and optionally the amount of the compound added to the receptacle, and make sure that the correct chemical compound, and optionally the correct amount thereof, is added into the correct receptacle into which the swab with the sample obtained from the patient's 103 body is to be inserted.

It is to be further noted that in such cases, where the user 102 is required to add one or more chemical compounds into the correct receptacle, the processing circuitry 105 can be further configured to perform one or more of the following:
(a) verify that the chemical compounds are not expired, e.g. using a method similar to the one disclosed herein with reference to FIG. 6, mutatis mutandis;
(b) verify that the chemical compounds are added to the receptacle during a pre-defined time window starting when the swab with the sample obtained from the patient's 103 body is inserted into the correct receptacle;
(c) store the chemical compounds addition date in association with a given receptacle into which the chemical compounds are added. This enables setting an expiration date that is a result of the addition of the chemical compounds into the correct receptacle, and such expiration date can be monitored in a similar manner to the sample expiration date monitoring described herein with reference to FIG. 6, mutatis mutandis;
(d) verify that the user 102 mixed the receptacle so that the added compounds are mixed with any existing compound present within the receptacle.

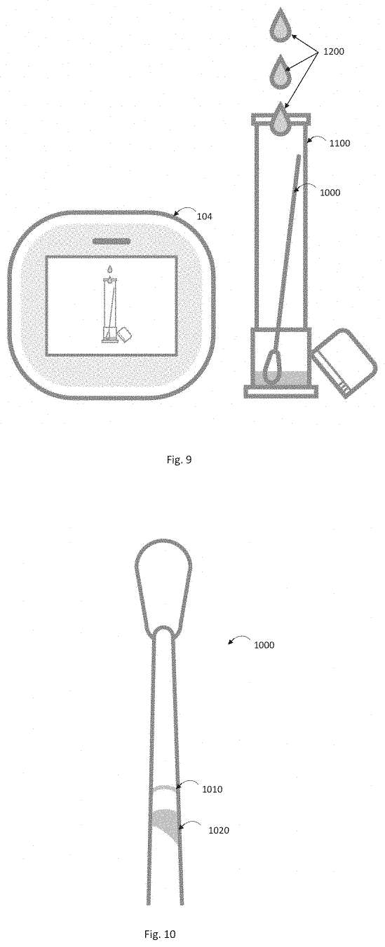

Attention is drawn in this respect to FIG. 9, showing a receptacle 1100, in which a swab 1000 is placed, and into which a chemical compound 1200 is added by a user 102. In the example illustrated in FIG. 9, a camera of a medical data acquisition device 104 is utilized to acquire images and/or video recording of the chemical compound addition in order to perform the above-mentioned verifications.

It is to be noted that in some cases one or more of the receptacles does not a-priori include any reagent or chemical compound, and the user 102 is required to add, into an identifiable receptacle (e.g. identifiable using a barcode or any other means/mechanism), one or more chemical compounds that are required for generating the required chemical reaction when the sample is inserted into the identifiable receptacle. In such cases, the processing circuitry 105 can be configured to analyze the chemical compound addition images and/or video recording to verify that the correct mixture of chemical compounds is inserted into the identifiable receptacle, optionally in a required order of addition of the various chemical compounds, and optionally in a required timing of addition of the various chemical compounds.

In some cases, the chemical compounds may be added to the receptacle in various manners, such as for example but not limited to, by breaking a capsule accommodating the chemical compounds and pouring thereof into the receptacle or breaking one or more capsules comprised by the receptacle. The mixture of the chemical compounds inside the receptacle may be monitored by the medical data acquisition device 104, e.g. by acquiring images and/or video recordings via a camera (being one of the sensors 106, or an external camera 110 external to the medical data acquisition device 104 and movable irrespective thereto) to verify that the mixture process is done correctly. The mixture process may be for example stirring the swab inside the receptacle for a predetermined number of times or for a predetermined duration of time, shaking the receptacle for a predetermined number of times or for a predetermined duration of time, before inserting the swab therein, in order to ensure that the chemical compounds were properly added and/or mixed therein, dipping the swab into the receptacle predetermined number of times. For this purpose, the processing circuitry 105 can be configured to analyze said images and/or video recordings of the mixture process to verify if this step was done correctly. The verification can be based on predetermined parameters, such as for example but not limited to, change in color of the chemical compounds inside the receptacle and/or change in liquid level inside the receptacle (e.g. by identifying the change in liquid level indicated by line markings on the swab/receptacle), angle of the swab after placing thereof inside the receptacle, immersion of the swab into the chemical compounds accommodated by the receptacle (e.g. to verify that the swab was immersed deep enough the chemical compounds), the amount of chemical compounds added to the receptacle (e.g. by monitoring the change in liquid level of the chemical compounds inside the container thereof), etc.

For this purpose, the medical data acquisition device 104 may be configured to monitor and verify each step of the mixture process, e.g., by utilizing a camera (being one of the sensors 106, or an external camera 110 external to the medical data acquisition device 104 and movable irrespective thereto) to verify that the correct chemical compounds are to be used (that is, the user 102 can acquire one or more images and/or video recordings of the chemical compound container that he would like to use and the processing circuitry 105 can be configured to analyze these images and/or video recordings in order to determine if a correct chemical compound container was chosen by the user 102).

The processing circuitry 105 can be further configured to perform one or more additional actions based on results of the analysis. In some cases, the additional actions may include one or more of:

(a) display a chemical compound container image on a display with a marking of the chemical compound container to be used for mixing/adding the chemical compound therein thereby indicating the user 102 the correct chemical compound container;

(b) upon the results indicating that the correct chemical compound container is chosen provide the user with a successful chemical compound container indication;

(c) upon the results indicating that the chemical compound container is not the correct chemical compound container provide the user with a failure chemical compound container indication or with chemical compound container instructions for choosing a correct chemical compound container.

In some cases, if incorrect chemical compounds were added to the correct receptacle, the processing circuitry 105 may instruct the user 102 to repeat the process collecting the sample from patient's 103 body using a new swab.

In some cases, after adding the chemical compounds inside the receptacle, the user 102 may be required and/or guided, e.g., by the medical data acquisition device 104, to perform specimen collection from the receptacle. The specimen collection may be for example and without limitation, collecting via a pipette a required amount of the mixture that is inside the receptacle, dipping and/or immersing a Litmus strip (that can be a Litmus paper, gel or any other suitable polymer) into the receptacle. Optionally, in such cases the user may be guided to perform additional one or more steps. For example, the user 102 may be guided, e.g., by the medical data acquisition device 104, to expose the receptacle and/or the collected specimen, e.g., via the pipette and/or the Litmus strip, to a UV light and/or heat, etc.

In some cases, the user 102 may be required and/or guided, e.g., by the medical data acquisition device 104, to perform additional steps, on the swab before and/or after collecting a sample and/or on a chemical compound container (e.g., before adding the chemical compound to the receptacle) and/or on the receptacle after adding the chemical compounds into the receptacle. The additional actions may be, for example and without limitation, exposing the swab and/or the receptacle and/or the chemical compound container to a UV light and/or heat, etc.

In some cases, for this purpose, the medical data acquisition device 104 may be configured to monitor and verify each step of the specimen collection process, e.g., by utilizing a camera (being one of the sensors 106, or an external camera 110 external to the medical data acquisition device 104 and movable irrespective thereto) to verify that the specimen is collected correctly (that is, the user 102 can acquire one or more images and/or video recordings of the collected specimen and/or of the whole specimen collection process and the processing circuitry 105 can be configured to analyze these images and/or video recordings in order to determine if the specimen was collected correctly by the user 102).

The processing circuitry 105 can be further configured to perform one or more additional actions based on results of the analysis. In some cases, the additional actions may include one or more of:

(a) display a specimen collection process image or video on a display with a marking of the relevant items (e.g., a pipette) to be used for adequately collecting the specimen and demonstrating steps to be taken during the specimen collection process;

(b) upon the results indicating that the specimen collection process was done correctly provide the user with a successful specimen collection process indication;

(c) upon the results indicating that the specimen collection process was not done correctly provide the user with a failure specimen collection process indication.

In some cases, if the specimen collection process was done incorrectly, the processing circuitry 105 may instruct the user 102 to repeat the process for specimen collection or repeat the process of collecting the sample from patient's 103 body using a new swab.

In addition, in some cases, ambient conditions may also be monitored by the medical data acquisition device 104, such as for example but not limited to, ambient temperature, light intensity, UV intensity or any other ambient parameters that may be related to one or more processes described herein, in accordance with the presently disclosed subject matter.

It is to be noted that in some cases the medical data acquisition device 104 can be configured to identify the required reagent or chemical compound that is required to be added to the receptacle, prior to its addition therein. In such cases, the processing circuitry 105 can be configured to analyze images, of chemical compound container chosen by the user 102, acquired by a camera (being one of the sensors 106, or an external camera 110 external to the medical data acquisition device 104 and movable irrespective thereto) to verify that the correct container is to be used (that is, the user 102 can acquire one or more images and/or video recording of the container that he would like to use and the processing circuitry 105 can be configured to analyze these images and or video recording in order to determine if a correct container was chosen by the user 102). The chemical compound container may be any container known in the art that is suitable for storing and/or containing chemical substances (e.g. liquid chemical solutions/additives, powder chemicals, etc.) therein, such as for example but not limited to, media bottles or graduated bottles, tinted amber (actinic) bottles that are typically used for protecting light-sensitive chemical compounds from visible light, ultraviolet and infrared radiation which may alter them or Boston round bottles. For the purpose of identifying the container, each container may have a unique identifier, such as for example but not limited to, a rubber stopper having a unique color indicating the chemical compound accommodated by the container, a barcode, a snap-top seal, a unique shape and/or color(s) of the container, a unique shape and/or color(s) of one or more portions of the container, a label located on the container and/or on a corresponding lid thereof, a Radio-frequency identification (RFID) and/or any combination(s) thereof and/or any other identifier that may be suitable for enabling unique identification in accordance with the presently disclosed subject matter.

It is to be further noted that in case any verification discussed herein fails—the processing circuitry 105 can provide a suitable notification to the user 102 and/or mark the sample as invalid so that it is not used for diagnosing a medical condition of the patient 103. Said notification may be provided, for example and without limitation, via a visual illustration (e.g. an image with appropriate instructions/illustrations therein presented on a screen of the medical data acquisition device 104 and/or a smartphone) and/or audio instructions (e.g. explanation on how to perform certain steps in accordance with the presently disclosed subject matter, provided for example by the medical data acquisition device 104 and/or a smartphone). Optionally, in some cases, said notifications may indicate next steps to be taken by the user 102, in accordance with the presently disclosed subject matter.

It is to be still further noted that in some cases, the chemical compounds to be added to the receptacle, the amount of each chemical compound to be added into the receptacle, the order of addition of the chemical compounds into the receptacle, the timing between addition of each chemical compound into the receptacle, the expiration dates, and any other parameter that is considered, may be defined by a protocol that can optionally be stored in a central location and can optionally be updated occasionally.

Generally, it is yet to be further noted that throughout the presently disclosed subject matter, images acquired by a camera (being one of the sensors 106, or an external camera 110 external to the medical data acquisition device 104 and movable irrespective thereto) may be video recordings, mutatis mutandis. Optionally, in some cases, the medical data acquisition device 104 may be configured to transmit the acquired images and/or video recordings, e.g., to a specific medical practitioner location 120 (e.g. via communication network 116 such as the Internet). Additionally or alternatively, the acquired images and/or video recordings, may be stored in data repository 107 of the medical data acquisition device 104.

The processing circuitry 105 can be further configured to perform one or more second actions based on results of the analysis (block 530).

In some cases, the second actions can include one or more of:

(a) display the sample placement image on a display with a marking of the given receptacle on the sample placement image thereby indicating the user 102 the correct receptacle;

(b) upon the results indicating that the swab is within the given receptacle provide the user with a successful receptacle placement indication;

(c) upon the results indicating that the swab is not within the given receptacle provide the user with a failure tube placement indication or with placement instructions for placing the swab within the given receptacle, wherein the tube can be for example and without limitation, a test tube, a glass, a cassette, a cuvette or any other type of receptacle into which the swab can be inserted;

(d) upon the results indicating that the swab is within the given receptacle provide the user with a medical diagnosis; or

(e) upon the results indicating that the swab is within the given receptacle provide the user with treatment instructions.

In some cases, if the swab was not placed within the correct receptacle the processing circuitry 105 can instruct the user 102 to repeat the process collecting the sample from patient's 103 body using a new swab.

It is to be noted that in some cases, upon placement of the swab, used during the process for collecting sample from patient's 103 body, within a correct receptacle and/or upon completion of the process for collecting sample from patient's 103 body and/or upon completion of the mixture/addition of the chemical compounds into the receptacle (e.g., as detailed herein above, inter alia, with respect to FIG. 9), the medical data acquisition device 104 does not provide the user 102 with a medical diagnosis. In such cases, the medical diagnosis can be provided for example by medical practitioner 124 that can be located at a medical practitioner location 120, remote from the patient location 100. It is to be noted in this respect that in some cases the receptacles may be designed in a manner that prevents the user 102 and/or patient 103 from viewing any change caused to the reagent comprised within the receptacles and/or to the specimen collected (e.g., via a pipette, a Litmus strip, etc.), so that the user 102 and/or patient 103 cannot perform self-diagnosis.

In other cases, the user 102 can notice some changes on the swab, used during the process for collecting sample from patient's 103 body, before or after placing the swab within a correct receptacle (e.g. color change of the tip of the swab, line indicators appeared or changed, etc.) and/or changes caused to the reagent comprised within the receptacles and/or changes caused to the specimen collected (e.g., via a pipette, a Litmus strip, etc.). Additionally, or alternatively, in some cases the user 102 and/or patient 103 can notice some changes in the receptacle in which the swab is placed (e.g. as a result of a chemical reaction of the reagent within the receptacle). It is to be noted that in some cases, even when changes are visible to the user 102 and/or patient 103, the medical data acquisition device 104 does not provide the user 102 with a medical diagnosis. The medical diagnosis can be provided for example by medical practitioner 124 that can be located at a medical practitioner location 120, remote from the patient location 100.

In some cases, the medical diagnosis can be provided based on the at least one image acquired during the process for collecting sample from patient's 103 body indicating a contact area wherein the sample was collected, optionally combined with clinical laboratory results of the collected sample.

It has been indicated herein that in some cases the medical data acquisition device 104 does not provide medical diagnosis to the user 102 and/or patient 103. It is to be noted, however, that in other cases, the medical data acquisition device 104 can provide a medical diagnosis to the user 102 and/or patient 103. In such cases, the medical data acquisition device 104 can monitor changes in the receptacle in which the swab is placed and/or changes in the specimen collected (e.g., via a pipette, a Litmus strip, etc.) and provide a diagnosis based on identified changes (e.g. a change in color of the reagent within the receptacle and/or specimen container, line indicators appeared or changed thereon, appearance of one or more symbols, etc.). The monitoring may be performed by utilizing a camera (being one of the sensors 106, or an external camera 110 external to the medical data acquisition device 104 and movable irrespective thereto) to acquire one or more images and/or video recordings of the changes in the receptacle in which the swab is placed and/or changes in the specimen collected and analyzing thereof via the processing circuitry 105. It is to be noted that in some cases the changes may be invisible to the human eye, but the medical data acquisition device 104 can view the changes, e.g. using special lighting (e.g. infrared, ultra violet, etc.), and upon identifying the changes, it can provide a diagnosis, e.g. based on diagnosis rules.

In some cases, when a diagnosis is provided, the user 102 can be provided for example and without limitation, with a percentage of accuracy for the diagnosis, one or more actions to be performed (e.g., to call a doctor), an internet link to a detailed information regarding the diagnosis, direct link to a doctor (e.g., a video call or a chat).

In some cases, when a diagnosis is provided indicating that the patient 103 has a medical condition that is infective, such diagnosis can be utilized in order to inform other patients that may have been infected by the infective medical condition of the patient 103. Such other patients can be identified, for example, as disclosed in co-pending PCT Patent Application No. PCT/IL2020/050138, which is incorporated herein, in its entirety, by reference. Additionally or alternatively, when a diagnosis is provided indicating that the patient 103 has a medical condition that is infective, such diagnosis may be transmitted, by the medical data acquisition device 104 (e.g. via communication network 116 such as the Internet), to e.g., a specific medical practitioner location 120. In some cases, the medical diagnosis may be provided or not provided in accordance with the respective protocol that may be associated with the diagnostic test type that was taken. For example, protocol associated with a COVID-19 test, wherein a nasopharyngeal sample was collected by the tip of the swab, may specify steps/actions to be taken, e.g. by the user 102 and/or the medical data acquisition device 104, once the sample was collected (e.g., placing the swab within a given receptacle and performing the process of addition of given chemical compounds therein). If the protocol does not specify that a medical diagnosis should be provided upon completion of certain steps, for example for COVID-19 test, then the medical data acquisition device 104 does not provide the user 102 with the medical diagnosis.

It is to be noted that in some cases the medical data acquisition device 104 can also monitor the time that lapsed since the user 102 obtained the sample from the patient's body until placement of the sample within the suitable receptacle. In some cases, the sample has to be inserted into the receptacle within a certain predetermined time from obtaining the sample (e.g. within a few seconds, minutes, hours, etc., optionally depending on the sample type and/or the reagent type within the receptacle into which the sample is to be inserted). In such cases, the medical data acquisition device 104 can verify that the collected sample is inserted into the suitable receptacle on time (e.g. before the predetermined time passes).

In some cases, the medical data acquisition device 104 can also monitor the time that lapsed since the first chemical compound was added into the receptacle until placement of the sample obtained from patient's body within the suitable receptacle.

In other cases, the medical data acquisition device 104 can also monitor the time that elapsed between adding one or more chemical compounds into the receptacle, during the mixture process described hereinabove (that is, if more than one chemical compound should be added into the receptacle, each time window that elapsed between each addition of successive chemical compounds, may be monitored by the medical data acquisition device 104 in order to assure that the addition of the chemical compounds into the receptacle is done correctly within predetermined time windows).

In some cases, the medical data acquisition device 104 can also monitor the time that elapsed between adding/mixing one or more chemical compounds into the receptacle and performing next action as described hereinabove (e.g. exposing the swab and/or the receptacle and/or the chemical compound container to a UV light and/or heat, etc.).

In some cases, the medical data acquisition device 104 can also monitor the maximal time that has elapsed for a given stage. For example, after the mixture process and placement of the swab inside the receptacle, the user may be guided to immerse a litmus strip into the mixture of the chemical compounds comprised by the receptacle. The soaking time of the litmus strip may be monitored by the medical data acquisition device 104 in order to ensure that it is not being soaked for a duration above the maximal time so that the litmus strip is not damaged. Alternatively, or additionally, once the litmus strip was removed from the receptacle, the maximal allowed time that elapsed from removing thereof until the indicated results (e.g. markings) are no longer adequate/valid (e.g. in some cases upon expiry of 10 minutes, from removing the litmus strip from a liquid, the markings on the litmus strip start to smear and therefore cannot adequately indicate the results) may also be monitored by the medical data acquisition device 104.

Attention is now drawn to FIG. 6, a flowchart illustrating an example of a sequence of operations carried out for expiration date validation, in accordance with the presently disclosed subject matter.

According to the presently disclosed subject matter, medical data acquisition device 104 can be configured to perform an expiration date validation process 600. For this purpose, the processing circuitry 105 can be configured to operate a camera (being one of the sensors 106, or an external camera 110 external to the medical data acquisition device 104 and movable irrespective thereto) to acquire at least one expiration date validation image (block 610). The expiration date validation image includes one or more receptacle, each having an identifier enabling determination of an expiration date of the respective receptacle. The identifier can be an expiration date printed on the receptacle and/or a barcode or another identifier identifying the respective receptacle based on which the expiration date can be obtained, e.g. by retrieving it from a data repository that can optionally be remote.

Processing circuitry 105 can be configured to analyze the acquired expiration date validation image to validate an expiration date of the receptacles (block 620). For example, the image can be analyzed to identify the identifier and determine the expiration date of the receptacles. The expiration date can be compared with a current date, and if the expiration date is after the current date—the receptacle is not expired and can be safely used, whereas if the expiration date is before the current date—the receptacle is expired and cannot be used.

Upon the expiration date of one or more of the receptacles indicating expiry, the processing circuitry 105 can be configured to perform an expiration action (block 630). The expiration action can include providing an expiration notification to a user 102. The user 102 can be instructed to obtain a new receptacle instead of the expired receptacle. In case a prescription is required, the processing circuitry 105 can be configured to automatically generate a prescription, or initiate a process for obtaining a prescription from a person authorized to issue such prescription (e.g. medical practitioner 124).

It is to be noted that the chemical compounds may also have an expiration date that may indicate the date after which the chemical compounds should not be used, e.g., for placing thereof inside the receptacle, and such expiration date may be monitored in a similar manner to the sample expiration date monitoring described hereinbelow with respect to FIG. 6, mutatis mutandis.

Turning to another aspect of the presently disclosed subject matter, it is to be noted that in some cases, the expiration date of a collected sample collected from the patient's 103 body and placed within a receptacle can be monitored. In some cases, samples are collected by a user of the medical data acquisition device 104 (for example and without limitation, the samples may be one or more swabs that were used for collecting a sample, one or more receptacles with swabs comprised therein, one or more specimens collected e.g., via pipette and/or the Litmus strip), and are then sent to another location (e.g. a laboratory, an HMO, a hospital, or any other location having suitable facilities) for analysis and/or diagnosis purposes. Transporting the receptacle takes time and it is required to monitor that the receptacles (that include the samples obtained by the user 102) arrive at their destination where they are processed within a certain predetermined time after the samples are obtained and places within the receptacles, to make sure that the samples that are placed within the receptacles are not expired. It is to be noted that the predetermined time may vary depending on the type of the sample collected from the patient's 103 body, the type of reagent in the receptacle, etc.

In some cases, the medical data acquisition device 104 may be configured to monitor a packaging process, that may be performed for example by the user 102, after collecting a sample and/or after mixing/adding the chemical compounds into the receptacle and/or after collecting a specimen. The packaging process may include choosing a correct transportation package, placing the correct item (e.g., one or more swabs, receptacles or specimens) therein, placing a correct label and/or seal on the transportation package, or performing any other action that may be required and/or guided by the medical data acquisition device 104. Optionally, each step of the packaging process may be based on an appropriate protocol. The protocol may comprise information regarding various packaging procedures, wherein each packaging procedure may be associated with the particular diagnostic test that was taken. Said protocol may be occasionally replaced or updated with new or additional packaging procedures/regulations (e.g. the procedures may correspond to new medical developments or can be based on a medical data retrieved from other medical data acquisition devices). In some cases, said changes to the protocol may be performed remotely (e.g. by downloading an update to the medical data acquisition device 104 via the internet). The transportation package may, be for example and without limitation, compliant mailing packs, absorbent sheets, flexible pouches, boxes, mailing bags and/or envelopes, specimen bags, polypropylene slide mailers, transport tubes, custom made sample transport packs, temperature protected shipping boxes, and/or any other sample transport packages that are compliant with the relevant regulations for transporting biological samples. Each package may have a unique physical and/or visual identifier(s) thereon in order to enable an appropriate identification thereof, for example, by the user 102 and/or by the medical data acquisition device 104, where applicable, in accordance with the presently disclosed subject matter. Said identifier(s) may be for example and without limitation, package shape and/or color, marks, color marks and/or labels, barcodes, RFIDs. The medical data acquisition device 104 may be configured to monitor and verify each step of the packaging process, e.g., by utilizing a camera (being one of the sensors 106, or an external camera 110 external to the medical data acquisition device 104 and movable irrespective thereto) to verify that the correct transportation package is to be used (that is, the user 102 can acquire one or more images and/or video recordings of the transportation package that he would like to use and the processing circuitry 105 can be configured to analyze these images and/or video recordings in order to determine if a correct transportation package was chosen by the user 102).

The processing circuitry 105 can be further configured to perform one or more additional actions based on results of the analysis. In some cases, the additional actions may include one or more of:

(a) display a transportation package image on a display with a marking of the transportation package to be used for placing the sample/receptacle/specimen therein thereby indicating the user 102 the correct transportation package;

(b) upon the results indicating that the sample/receptacle/specimen is within the given transportation package provide the user with a successful transportation package placement indication;

(c) upon the results indicating that the sample/receptacle/specimen is not within the given transportation package provide the user with a failure transportation package placement indication or with placement instructions for placing the sample/receptacle/specimen within the given transportation package.

In some cases, the medical data acquisition device 104 may be configured to monitor package transportation stages. For example, packaging/shipping/successful delivery time and date may be stored and monitored by the processing circuitry 105, and optionally the user 102 may be notified during the transportation of the package (e.g., the user 102 may be notified once the package was collected and/or successfully delivered to its destination).

Referring back to the monitoring of the expiration date of a collected sample collected from the patient's 103 body, the medical data acquisition device 104 can be configured to store a sample collection date in association with a given receptacle into which a sample is inserted by the user 102. In some cases, the sample collection date is determined automatically, e.g. as part of the sample placement verification process 500, so that upon verifying that the sample was placed in the correct receptacle, the date of such placement is recorded. It is to be noted that in some cases, the sample collection date can be provided by the user 102 manually, e.g. via the patient workstation 114. In other cases, the sample collection date can be provided by a medical practitioner 124 that is viewing the sample collection process in real-time. In such cases, medical practitioner 124 can manually record the sample collection date or it can be automatically identified by the medical practitioner workstation 122.

Upon a receptacle being analyzed at its analysis location (e.g. for diagnosis purposes), it can be identified (e.g. utilizing a barcode or any other identifier that enables identifying the receptacle) and it's sample collection date can be retrieved from the data repository on which it is stored (e.g. by a computer located at the analysis location). The time that passed since the sample collection date can be determined and compared (e.g. by a computer located at the analysis location) with an expiration date that is determined for that sample (e.g. based on the type of the sample collected from the patient's 103 body, the type of reagent in the receptacle, etc.). In case the sample collection date indicates that the sample is expired—a notification can be issued, to a medical practitioner 124 of the patient 103, and/or to the patient 103, and/or to the user 102, indicating that the sample collection should be repeated.

It is to be noted that when reference is made herein to sample collection date and/or specimen collection date, it can include a date and a time of collection of the sample/specimen and/or placement thereof in the respective receptacle/pipette/Litmus strip/etc.

It is to be further noted that in some cases, medical data acquisition device 104 and/or patient workstation 114 can be configured to provide the user 102 and/or patient 103 with treatment recommendations and/or prescriptions upon diagnosing a medical condition that requires treatment. The treatment recommendations and/or prescriptions can be determined based on pre-determined rules that depend on the diagnosis of the medical condition of the patient 103 based on analysis of the changes in the receptacle in which the swab comprising a sample collected from the patient's 103 body is placed (e.g. as detailed herein, inter alia with reference to FIG. 5). In some cases, the treatment recommendations and/or prescriptions are determined by the central system 130 and/or by the medical practitioner workstation 122, based on the information obtained by the medical data acquisition device 104 (e.g. images of the receptacle in which the swab comprising a sample collected from the patient's 103 body is placed) and sent thereto.

In some cases, various processes described herein, inter alia, expiration date monitoring, expiration date validation, sample collection process, sample placement verification process, addition of chemical compounds (e.g., the amount added, type of chemical compound used, order of addition, time elapsed between the additions, etc.), chemical compound mixture process, navigation process, specimen collection process, packaging process, and/or specific steps/stages/actions taken during one or more of said processes and/or combinations thereof may be based on a predetermined protocol. The protocol can comprise information regarding various procedures and/or specify a correct order of steps/stages/actions to be taken, wherein each may be associated with the particular diagnostic test type to be taken. Said protocol can be occasionally replaced or updated with new or additional procedures (e.g. the procedures can correspond to new medical developments or can be based on a medical data retrieved from other medical data acquisition devices). In some cases, said changes to the protocol can be performed remotely (e.g. by downloading an update to the medical data acquisition device 104 via the internet).

It is to be further noted that the medical data acquisition device 104 may be utilized for performing all or portions of the steps and/or processes described herein. For example, the medical data acquisition device 104 may be utilized merely for monitoring the sample preparation and/or placement processes (e.g., monitoring and/or validating the collected sample placement inside the receptacle or monitoring and/or validating the mixture process inside the receptacle). In such cases, the sample collection process may not be monitored by the medical data acquisition device 104 and only one or more of the succeeding steps may be monitored thereby.

It is to be also noted that the medical data acquisition device 104 may be configured to order replacement items during or after performing one or more steps and/or processes described herein. For example, new swabs may be ordered in case the existing swabs have expiration date that has elapsed. It is to be noted that the term “machine-readable storage medium” should be taken to include a single medium or multiple media (e.g. centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present presently disclosed subject matter. the term “machine-readable storage medium” shall accordingly be taken to include, but not limited to, solid-state memories, and optical and magnetic media.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

Examples of the presently disclosed subject matter may be provided as a computer program product, or software, that may include a machine-readable medium having stored thereon instructions, which may be used to program a computer system (or other electronic devices) to perform a process according to the presently disclosed subject matter. A machine-readable medium includes any mechanism for storing or transmitting information in a form readable by a machine (e.g. a computer). For example, a machine-readable (e.g. computer readable) medium includes a machine (e.g. a computer) readable storage medium (e.g. read only memory (“ROM”), random access memory (“RAM”), magnetic disk storage media, optical storage media, flash memory devices, etc.), a machine (e.g. computer) readable transmission medium (electrical, optical, acoustical or other form of propagated signals (e.g., infrared signals, digital signals, etc.)), etc.

In the foregoing specification, the presently disclosed subject matter has been described with reference to specific examples of embodiments of the presently disclosed subject matter. It will, however, be evident that various modifications and changes may be made therein without departing from the broader spirit and scope of the presently disclosed subject matter as set forth in the appended claims.

Also, the presently disclosed subject matter is not limited to physical devices or units implemented in nonprogrammable hardware but can also be applied in programmable devices or units able to perform the desired device functions by operating in accordance with suitable program code, such as mainframes, minicomputers, servers, workstations, personal computers, notepads, personal digital assistants, electronic games, and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

While certain features of the presently disclosed subject matter have been illustrated and described herein, many modifications, substitutions, changes, and equivalents will now occur to those of ordinary skill in the art. It is, therefore, to be understood that the appended claims are intended to cover all such modifications and changes as fall within the true spirit of the presently disclosed subject matter.

1. A medical data acquisition device comprising a processing circuitry, the processing circuitry configured to:
operate a camera to acquire at least one image during a process for collecting a sample by a tip of a swab making contact with a required contact area on, or within, a body of a patient;
analyze the image to determine one or more parameters, the parameters including at least one of (a) a position of the swab with respect to the required contact area, or (b) an orientation of the swab with respect to the required contact area; and
perform at least one action based on comparison of the one or more parameters with a positioning criterion.

2. The medical data acquisition device of claim 1 wherein the camera is comprised within the medical data acquisition device and wherein the medical data acquisition device further comprises a mechanical connection for connecting the swab so that images captured by the camera include at least a part of the swab upon the swab being connected to the medical data acquisition device via the mechanical connection.

3. The medical data acquisition device of claim 2, wherein the medical data acquisition device is a smartphone and wherein the mechanical connection is an add-on connectable to the smartphone for enabling positioning of the swab so that images captured by the camera include at least the part of the swab upon the swab being connected to the medical data acquisition device via the mechanical connection.

4. The medical data acquisition device of claim 1, wherein the swab includes one or more scale mark and/or graduation marks enabling determination of a penetration depth of the swab into a body cavity of the patient.

5. The medical data acquisition device of claim 1, wherein the swab includes one or more rotation determination markings enabling determination of rotation of the swab around an axis of rotation.

6. (canceled)

7. The medical data acquisition device of claim 1, wherein the medical data acquisition device further comprises a mechanical connection for connecting the swab and wherein the camera is external to the medical data acquisition device and movable irrespective of the swab upon the swab being connected to the medical data acquisition device via the mechanical connection.

8. The medical data acquisition device of claim 1, wherein upon the comparison of the one or more parameters with the positioning criterion indicating that the swab is not at a desired spatial position with respect to the required contact area, the action is one or more of: alerting a user of the medical data acquisition device, or marking the sample as invalid.

9. The medical data acquisition device of claim 1, wherein upon the comparison of the one or more parameters with the positioning criterion indicating that the swab is at a desired spatial position with respect to the required contact area, the action is one or more of: providing a user of the medical data acquisition device with a successful positioning indication, or marking the sample as valid.

10-14. (canceled)

15. The medical data acquisition device of claim 1, wherein the processing circuitry is further configured to:
operate the camera to acquire a sequence of images during the process for collecting the sample;
analyze the sequence of images to determine one or more navigation parameters, the navigation parameters including at least one of: (a) a navigation position of the swab with respect to the required contact area, or (b) a navigation orientation of the swab with respect to the required contact area; and
based on the navigation parameters, provide a user of the medical data acquisition device with navigation instructions for navigating the tip of the swab to contact the required contact area.

16-41. (canceled)

42. A method comprising:
operating, by a processing circuitry, a camera, to acquire at least one image during a process for collecting a sample by a tip of a swab making contact with a required contact area on, or within, a body of a patient;
analyzing, by the processing circuitry, the image to determine one or more parameters, the parameters including at least one of (a) a position of the swab with respect to the required contact area, or (b) an orientation of the swab with respect to the required contact area; and
performing, by the processing circuitry, at least one action based on comparison of the one or more parameters with a positioning criterion.

43. The method of claim 42, wherein the camera is comprised within a medical data acquisition device and wherein the medical data acquisition device comprises a mechanical connection for connecting the swab so that images captured by the camera include at least a part of the swab upon the swab being connected to the medical data acquisition device via the mechanical connection,

44. The method of claim 43, wherein the medical data acquisition device is a smartphone and wherein the mechanical connection is an add-on connectable to the smartphone for enabling positioning of the swab so that images captured by the camera include at least the part of the swab upon the swab being connected to the medical data acquisition device via the mechanical connection.

45. The method of claim 42, wherein the swab includes one or more scale mark and/or graduation marks enabling determination of a penetration depth of the swab into a body cavity of the patient.

46. The method of claim 42, wherein the swab includes one or more rotation determination markings enabling determination of rotation of the swab around an axis of rotation.

47. (canceled)

48. The method of claim 42, wherein the swab is connected to a medical data acquisition device via a mechanical connection and wherein the camera is external to the medical data acquisition device and movable irrespective of the swab upon the swab being connected to the medical data acquisition device via the mechanical connection.

49. The method of claim 42, wherein upon the comparison of the one or more parameters with the positioning criterion indicating that the swab is not at a desired spatial position with respect to the required contact area, the action is one or more of: alerting a user of the medical data acquisition device, or marking the sample as invalid.

50. The method of claim 42, wherein upon the comparison of the one or more parameters with the positioning criterion indicating that the swab is at a desired spatial position with respect to the required contact area, the action is one or more of: providing a user of the medical data acquisition device with a successful positioning indication, or marking the sample as valid.

51. The method of claim 42, wherein the required contact area is within a cavity of the body and wherein the positioning criterion depends on a length of an inserted part of the swab inserted into the cavity.

52-55. (canceled)

56. The method of claim 42, further comprising:
operating, by the processing circuitry, the camera to acquire a sequence of images during the process for collecting the sample;
analyzing, by the processing circuitry, the sequence of images to determine one or more navigation parameters, the navigation parameters including at least one of: (a) a navigation position of the swab with respect to the required contact area, or (b) a navigation orientation of the swab with respect to the required contact area; and
based on the navigation parameters, providing, by the processing circuitry, a user of the medical data acquisition device with navigation instructions for navigating the tip of the swab to contact the required contact area.

57-82. (canceled)

83. A non-transitory computer readable storage medium having computer readable program code embodied therewith, the computer readable program code, executable by at least one processing circuitry to perform a method comprising:
operating, by the processing circuitry, a camera, to acquire at least one image during a process for collecting a sample by a tip of the swab making contact with a required contact area on, or within, a body of a patient;
analyzing, by the processing circuitry, the image to determine one or more parameters, the parameters including at least one of (a) a position of the swab with respect to the required contact area, or (b) an orientation of the swab with respect to the required contact area; and
performing, by the processing circuitry, at least one action based on comparison of the one or more parameters with a positioning criterion.

84. (canceled)
